(19) United States
(12) Patent Application Publication
(10) Pub. No.: US 2016/0120691 A1
(43) Pub. Date: May 5, 2016

(54) NORMOTHERMIC MAINTENANCE METHOD AND SYSTEM

(71) Applicants: Laurence KIRWAN, New York, NY (US); Stephen MCLOUGHLIN, Hampshire (GB)

(72) Inventors: Laurence KIRWAN, New York, NY (US); Stephen J. MCLOUGHLIN, Hampshire (GB)

(21) Appl. No.: 14/890,233

(22) PCT Filed: May 9, 2014
(86) PCT No.: PCT/EP2014/059528

§ 371 Date: Nov. 10, 2015

Related U.S. Application Data
(60) Provisional application No. 61/855,220, filed on May 10, 2013.

(51) Int. Cl. A61F 7/02; A61F 7/03

(52) U.S. Cl. CPC A61F 7/02; A61F 7/03; A61F 2007/0036

(57) ABSTRACT

A method of maintaining advantageously stabilized homeostatic parameters through normothermic maintenance in particular in the peri-surgical environment is described. In particular, the method of advantageously applying insulative garments over a small but disproportionately effective percentage of the BSA in order to counter environmentally and surgically induced hypothermia. Further described is a method of maintaining normothermia using a disposable or re-usable, un-tethered, anatomically formed or substantially tube formed garment or plurality of garments preferentially configured to enclose the extremities and in particular the areas of the mammalian body which are the most effective loci of heat transfer. The method, whereby the positive heating attributes of the system may be augmented through the use of additional insulative garments, which garments may be disposed about the patient's body to achieve greatest benefit to both patient and surgical field.

The instant invention relates generally to surgery and more specifically to a method, apparatus, and system for maintaining normothermic conditions in a patient before, during and after surgery. In summary the invention, comprises a low cost, self-contained, untethered, preferably disposable and recyclable garment which is formed to be worn at the extremities of the patients' limbs. Advantageously, the device can be worn in conjunction with standard surgical garments, which may incorporate passive insulation methods, thereby enhancing the overall system efficiency.

### BACKGROUND

The instant invention is of common ownership with US Patent Filing 2012/61690523 filed 28 Jun., 2012 “Normothermic Maintenance System & Method”.

As with the prior invention, ibid, the instant invention relates to improvements to methods and systems for maintaining patient euthermia, particularly during surgical intervention. One objective of the invention is to reduce patient morbidity during surgical intervention while beneficially expediting patient flow in the hospital environment. Yet another objective is the advantageous application of heat via independent systems and methods. Yet a further objective is the improvement of the economics of surgical treatment by the use of either disposable or re-usable simple normothermic maintenance systems. This particular device fulfills the advantageous elements of the prior invention, while extending its applicability.

Whereas the prior invention was a self-contained normothermic system which sought to improve patient wellbeing for longer duration operations, the instant device can be used in conjunction with existing hospital garments in order to provide a normothermic environment for shorter duration, outpatient operations and operations where the normothermic heat requirement is reduced.

The inventors believe that prior art in this domain fails to adequately address the specific practicalities of the surgical environment which failure significantly contributes to the development of patient hypothermia. The inventors intend that the instant method and system acts to maintain normothermia rather than allowing patient hypothermia to establish itself and then seek to redress the hypothermic condition. The inventors further consider and will show that heating the extremities rather than heating the torso is a more practical solution for the prevention of hypothermia.
Maintain normothermia in the patient rather than correct for hypothermia
Reduce morbidity through maintenance of normothermia
Improve operating room environment
Intervention-less garment
Application of heat to the extremities.
Low cost, disposable and re-usable.

In the field of surgery it is recognized that maintaining the patient at a euthermic temperature has both a therapeutic and a comfort value. Historically the therapeutic value largely went unrecognized and the comfort value of the patient was subordinated to the comfort of the surgeon, presumably on the basis that the patient was unconscious.

What is now recognized is that application of hypothermia as a deliberate element of the surgical procedure should be restricted to zonal surgeries where particularly sensitive tissue damage may result from normal or elevated corporeal temperatures such as in cardiac surgery or neurosurgery.

Christensen et al U.S. 2011/0172749 define and confirm a traditional perspective on hypothermia and the inter-relationships between different elements of the circulatory system: Hypothermia . . . , is the result of prolonged exposure to a cold challenge where blood flow through the venous plexuses and AVA's can be near zero of the total cardiac output. “Hypothermia . . . is the result of prolonged exposure to a cold challenge where blood flow through the venous plexuses and AVA's can be near zero of the total cardiac output. Vasoconstriction of the peripheral blood vessels may arise under hypothermia in order to prevent further heat loss by limiting blood flow to the extremities and reducing heat transfer away from the thermal core of the body. However, vasoconstriction makes it much more difficult to reverse a hypothermic state since vasoconstriction impedes the transfer of heat from the body surface to the thermal core and makes it difficult to simply apply heat to the surface of the body. This physiological impediment to heat transfer is referred to as a vasoconstrictive blockage to heat exchange.”

The instant inventors, agree with the Christensen physiological analysis but contend that the effects of psychological stress is an additional, un-quantified, significant, yet understated, contributor to the early onset of hypothermia in the surgical environment.

In the surgical environment, the initial stages of hypothermia start to occur in the pre-operative period when the patient is asked to disrobe; this procedure typically occurs in a cool environment. The patient is then given a surgical garment. Surgical garments currently in use are not conducive to insulating the body, or to the maintenance of normal body temperature. They are often skimpy, poorly fitting, garments, open at the back, with minimal coverage of the patient's extremities and severely compromise the patient's modesty and self-respect. All of which contributes to increasing the patient's anxiety and heightening sensitivity to external stimuli in an unfamiliar environment. The patient is then left in an alien environment, partially clothed and potentially hypothermic which again results in a relatively high level of anxiety. The resultant increases to pulse rate, cardiac output and increasing secretion of adrenergic stress hormones such as adrenaline, cortisol and insulin are characteristics of a ‘fight and flight’ physiological response. It is well known that adrenaline is a potent vasoconstrictor and that peripheral vaso-constriction and redirection to the core is a standard physiological response to stress, which further aggravates the ability of the body to maintain a homeothermic environment and therefore predisposes the patient to hypothermia.

Practically, this physiological response also creates difficulties in inserting intra-venous (IV) cannulas in the ambulatory/pre-surgical holding area. IN addition patient hypothermia detrimentally compounds the difficulties of efficient monitoring of oxygen saturation in the operating room as pulse oximetry readings become compromised.

The inventors conclude that these environmental and physiological factors contribute to normothermic degradation prior to anaesthesia, while significantly increasing the difficulties of patient care.

The inventors therefore conclude that early stage hypothermia is being induced in the pre-operative process of preparing the patient for surgery both in the pre-surgical holding area and the operating theatre and that the negative impacts of the propensity to early onset of the hypothermic condition have not been fully appreciated.

In addition, incremental developments in surgical technique, driven by technological advances are changing the timing of patient throughput. Writing in the Journal of Anaesthesia 19(2), 2009: pp 190-4, Sasano et al report that significant increases in the time progression from the patient's operating room entrance to incision are being observed. These time delays are attributed to recent advances in surgical technology. The study reported that the time taken in 2008-2009 was 30.4 minutes (with a margin of error of +/−8.8 minutes) longer than the time for similar procedures in 1685-1686. Such delays are almost certain to have a significant negative impact on the patient's body temperature, health and wellbeing and the inventors seek to address this through the use of their invention.

The nominal temperature of the human body should be 37° C. with hypothermia being defined as a core temperature <36.0° C., although the U.S. Army define the upper threshold of hypothermia as being a core temperature of <35° C., possibly in response to the age and fitness of their corps of personnel. A normal and desirable core temperature state is referred to as ‘normothermia’. The condition of hypothermia is further sub-divided into mild, (32-35° C.), moderate, (21-31° C.) and deep (15-25° C.). Augustine et al 2002/0153813 confirms normothermic temperature limits for the body as being 25° C. +/−1° C. further teaching “ . . . surface tissue varies in temperature according to whereon the body it is located. The skin of the torso is usually hypothermic, while the skin of the legs is always hypothermic. The normal skin temperature of the distal leg of a patient is approximately 32° C., which is considered to be “moderately hypothermic”. The skin temperature of the distal leg of a patient with vascular insufficiency may be as low as 25° C., which is “severely hypothermic.” Augustine confirms the necessity for maintaining normothermia: “This extremely tight temperature control is necessary because virtually all cellular functions, chemical reactions and enzymatic reactions are optimum at normothermia”.

Industry literature widely acknowledges that, except in cases where tissue degeneration is deliberately arrested by cooling, hypothermia is generally detrimental to patient recovery and may put life at risk and this is reflected in a large number of learned publications.

A study by Karalapillai et al, (2016) published in Anaesthesia 66(9): 780-784, September 2016, which utilized an extensive dataset derived from surgical interventions conducted in Australia and New Zealand sought to correlate surgical risk and hypothermia and additionally to create definitions for transient and persistent hypothermia. The study was able to successfully correlate increased risk of death in elective cardiac surgery patients with persistent hypothermia. “Persistent hypothermia” was defined as a core temperature of less than 36° C. which endured for in excess of 20 hours. Karalapillai comments: “Transient hypothermia was not independently associated with increased hospital mortality (OR=0.9, 95% Cl 0.8-1.1), whereas persistent hypothermia was associated with markedly increased risk of death (OR=6.3, 95% Cl 3.3-171.0).” The inventors note that persistent hypothermia as defined by Karalapillai et al is only 1° C. lower than normothermic temperature.

Another study by Qadan et al, entitled “Hypothermia and Surgery: immunologic mechanisms for current practice and published in Annals of Surgery 250(1): 130-127, July, 2009, commenced, “Anesthetized surgical patients are particularly at risk for hypothermia, which has been directly linked to the development of sequelae, such as coagulopathy, infection, morbid myocardial events, and death after surgery,” they were further able to form the conclusion: “Hypothermia exerts multiple effects at the cellular level, which impair innate immune function, and are associated with increased septic complications and mortality.”

In a paper entitled “Effects of perioperative hypothermia and warming in surgical practice”, published in International Wound Journal, 2(3): 163-204, (2005) Kumar et al, reported: “Perioperative hypothermia is common and adversely affects clinical outcomes due to its effect on a range of homeostatic functions. Many of these adverse consequences are preventable by the use of warming techniques . . . . The primary beneficial effects of warming are mediated through increased blood flow and oxygen tension at tissue level. Reduction in wound infection, blood loss and perioperative pain with warming is promising . . . . Achieving normothermia throughout the patient's journey is a worthwhile goal in surgical patients.”

In addition to the clinical benefits of removing hypothermia, Defina et al in the Journal of PeriAnesthesia Nursing 13(4):189-195, August, 1698, noted that the removal of hypothermia was observed to result in improvements in the economics of patient health, concluding; “The inadvertent hypothermia that is often seen after anesthesia in a cool environment has been associated with delays in recovery and longer stays in the PACU . . . . Patients who arrived in the PACU hypothermic had longer PACU stays than patients who arrived normothermic.”

A decade later a further study was able to put statistics to Defina et al's conclusions. Writing in the periodical Best Practice and Research. Clinical Anaethesiology 18(4): 630-657, December 2008, Reynolds et al reported: “Only a 1.9° C. core hypothermia triples the incidence of surgical wound infection following colon resection and increases the duration of hospitalization by 20%. Furthermore mild hypothermia triples the incidence of postoperative adverse myocardial events. Thus, even mild hypothermia contributes significantly to patient care costs and needs to be avoided.” Clearly, in addition to patient welfare, there are significant financial implications to the removal of patient hypothermia.

The benefits of sustained normothermia in surgical patients have the gross effect of reducing the morbidity rate of surgical intervention. Predictable reduction of patient morbidity enhances patient wellbeing and patient throughput can thereby be increased with significant benefit to both patient and surgical centers. In summary, the effect of hypothermia on surgical patients is particularly extreme: the peri-operative environment of pre-surgical areas, operating rooms and post anaesthesia care units are typically maintained at lower than normal ambient temperatures. Traditional medical surgical garments fail to offer the necessary insulative construction or material properties to be able to maintain normothermic body temperature in this environment.

Finally, the process of anaesthesia, which reduces metabolic rate, further conspires to reduce body temperature.

Unfortunately, the autonomic defense reflex of shivering, which could at least temporarily or partially alleviate the hypothermic condition and by which the body indicates a requirement to increase core temperature is not available to the anaesthetized patient. Shivering generates between two and three times the normal metabolic thermal production. The patient's inability to shiver therefore serves to further reduce body core temperature and accentuate the onset of hypothermia. The maintenance of normothermia is therefore of further practical benefit to both patient and surgical team, as it reduces unwanted fluctuations in pulse rate, cardiac output and create an improved homeostatic platform and promote efficient wound perfusion.

However the effect of hypothermia extends beyond the immediate surgical environment. In an article, entitled, “Antibiotic prophylaxis in clean surgery: clean non-implant wounds” by Leaper et al, published in Journal of Chemotherapy, 13 Spec No 1 (1): 96-101, 2001 November, the correlation was drawn between reduced requirement for antibiotic prophylaxis and the maintenance of normative temperature during surgery, concluding: “Wound infection after clean surgery (the majority being hernia, varicose vein and breast surgery) is often greatly underestimated. If a trained and blinded observer is involved using close and prolonged surveillance . . . , an infection rate of 12% or more may be found. Equally controversial is the value of prophylactic antibiotics in preventing postoperative wound infection; there is no clear cut evidence of efficacy and some random controlled trials have shown no differences at all . . . . An alternative to antibiotics is the systemic warming of patients or the local warming of the operative site prior to surgery.”

Peri-operative heating of the patient is clearly considered advantageous. However, prior art in this field has created conflict between the practical application of existing garments and blankets and peri-operative surgical procedures which detrimental combination conspires to marginalize the efficacy of the heating process while simultaneously creating significant levels of additional complexity in the surgical environment. The current Forced Air Warming “FAW” devices, for example, seek to redress the problem of hypothermia in the pre-operative holding area by using an inflatable gown attached to a wall mounted electric powered device. This, aside from the necessity its removal for surgical marking, is then used intra-operatively for limited upper body and extremity coverage. Furthermore, existing FAW heat devices conflict with both temporal and spatial requirements of the surgical environment.

Further academic studies have been conducted which consider the negative impact which intra-operative heating devices have on the surgical environment and also to determine if pre-operative heating alone is sufficient to remove surgically induced hypothermia. These studies unanimously concluded that pre-surgical heating is insufficient and that the patient derives optimal benefit if artificial heat is also applied during the intra-operative and post operative phases.

It is therefore the inventors' intention to improve patient peri-operative warming and coincidentally create, through the use of their invention, an improved environment for the surgical team, particularly in short, i.e. less than 2 hours, and medium, from 2-6 hours duration surgical interventions. For purposes of clarification, operative duration is defined from when the patient enters the operating room to when the patient leaves the operating room. This should be distinguished from “surgical duration” which is measured from time between first incision (“knife to skin”) to completion of closure of the surgical incision. A further and alternative classification of peri-operative duration would be defined as the entire duration of the surgical admission from the time the patient enters the pre-surgical area to the time of discharge from recovery to home or to in-patient care.

The inventors note that existing devices preferentially consist of garments and blankets which inefficiently supply heat to the torso, whereas, the instant method and device focuses on the prevention of hypothermia through the provision of heat via gloves which contain heat elements which are used to provide a heat source which is both untethered and self-contained. For preference, the heat element is of a type which derives its energy from chemical reaction, although any other heat element which has heat output attributes of similar duration may equally be considered to be within the scope of the invention. The device may also comprise socks or bootees which contain heat elements and which further contribute an active heat source to the patient's extremities. For the sake of simplicity the device will be referred to as “gloves” unless further clarification is imparted by mentioning “socks” or “bootees”. As will be seen later, heating the hands and feet more effectively utilizes the body's structure to advantageously distribute heat throughout the corpus.

Examining the majority of existing patient heat devices reveals that these are tethered to both the patient and to fixed points located in the surgical environment. Such heating blankets and garments may require temporary removal prior to surgery, and additionally at one or more times during surgery. For example, in the pre-surgical environment, when the surgical site is marked by the operating surgeon thereby necessitating the heat garment removal. The inventors observe that the removal and replacement of existing surgical warming devices creates opportunity for the patient to become hypothermic while requiring additional, undesirable, time-consuming and costly intervention by skilled nursing staff and they seek, through the deployment of their invention, to eliminate this unnecessary element in the surgical procedure to the advantage of both patient and hospital staff.

The inventors feel that the advantages of their invention can most efficiently illustrated by making a direct comparison with existing technologies. When deploying the instant invention, for preference, the patient clothes him/herself in the surgical garments which are the preferred choice of the surgeon/surgical team and then puts on the gloves which are then activated to begin heating the patient. The premise behind this is that it is easier to maintain normothermia than combat hypothermia. Additionally, in using the instant invention in the pre-surgical environment, the devices require little or no additional intervention by nursing or medical-surgical personnel, thereby reducing the number of tasks to be completed by the surgical staff and simplifying the patient-care algorithm. These procedural advantages, which start in the pre-operative environment, continue for the duration of the surgical procedure and confer significant physiological, operational and economic benefits over prior art.

As the devices proposed by the instant invention are advantageously disposed on the extremities of the patient's limbs, this leaves the primary surgical area of the torso free and unencumbered. Indeed, it can be calculated that with both the patients' hands and feet covered and representing only 13% of the total body area, that, at a minimum, 87% of the body surface area remains unencumbered and free for surgical intervention. As will be illustrated, notwithstanding the small BSA which is covered by the device, the area which is covered is beneficially equipped to transmit heat. Thus, the proposed method advantageously provides quality heat distribution utilizing a smaller proportion of the body surface area, “BSA”, while leaving a larger proportion of the body free for surgical interventions, than is possible with existing systems and devices.

The torso, which is the traditional area for the placement of peri-surgical heating garments and covers represents only 32% of the human Body Surface Area, “BSA”, and is an area which incorporates significant thermal insulation in the form of fat and tissue layers. The extremities however, have a higher ratio of BSA to body mass/volume, which correlates with increased sensitivity to ambient temperature and accelerated heat loss. More importantly, the extremities of the limbs, although representing a smaller component of the human BSA contain better “quality” conductive tissues to which the application of heat may be advantageously made. The inventors therefore contend that the deployment of the instant invention yields heat absorption advantage to the patient which is equal to or better than existing heat technologies applied to the torso, or a portion of the torso. Heating the patient's limbs via heat transfer to AVA's and venous plexuses which are close to the skin surface, should be favorably compared and contrasted with heating the patient's torso. Therefore, by applying heat to an area of greater thermal sensitivity, heat transfer to the extremities of the patients' limbs acts to provide significant improvements to patient normothermia.

As a large percentage of the major organs which require surgical intervention are located on the torso, deployment of the instant invention does not interfere with the surgical site. Recent statistics from the American Society of Plastic Surgeons indicate that 36% of surgeries conducted in 2016 were conducted exclusively on the torso. This figure rises to 49% with the inclusion of non-specified liposuction. Thoracic and/or abdominal surgical operations typically allow only a portion of the torso to be covered during the procedure, which therefore reduces the effectiveness and applicability of existing heating devices. By comparison, as previously indicated, the instant invention allows surgical access to, at a minimum, 87% of the body surface area, which means that in surgical interventions which are located on the torso or upper limbs and where blanket type devices are inappropriate due to their interference with the surgical process, the instant device may be advantageously deployed. Furthermore, blanket type devices tend to apply zoned heating such that in the zones which are removed or de-activated there is, effectively, no patient heating and therefore significant potential for areas of the patient's torso to be subjected to significant heat loss. Such heat blankets typically also leave the patient's arms uncovered, thereby contributing to further increases in heat loss.

Additionally, blanket type devices provide unwanted heat and noise to the environment of the operating room, to the detriment of the surgical staff. On occasion existing torso-heating devices may be supplemented by additional cover for the arms, or for the legs, thereby increasing the effective coverage area. However, these devices are typically inflatable which increases the bulk of the patient in the surgical environment, encroaching on the available space in the operating room and creating significant practical and environmental difficulties for the surgical team.

The instant invention allows the active heating device to be worn in proximity to the skin surface as a garment which advantageously does not require additional securing to the patient. The instant device also serves to reduce complexity in the surgical environment as it does not require connection via by an umbilical attachment to an external heat and electrical source.

Turning now to the wider existing surgical environment, the majority of surgical devices deployed at present which promote normothermia require ancillary equipment. In one embodiment these take the form of hot air blowers which have the tendency to increase the ambient air temperature, to the detriment of the surgical staff. Such devices are also connected by umbilical conduits which serve to connect the patient to external heat or electrical sources. The umbilicals themselves create additional, unwanted complexity in the surgical environment and pose significant, attendant Health & Safety Hazards. The instant device, advantageously, does not require external attachments or umbilicals. Other embodiments which promote normothermia by heat transfer from circulating hot liquids around garments also disadvantageously require umbilicals and additional tethering. Therefore, yet another advantage of the instant device is that it does not require additional ancillary equipment, which may adversely impact the surgical environment.

Finally, prior art in this field includes devices which transmit heat by convection and radiation. The instant device more advantageously transmits heat to the patient predominantly by conduction and radiation.

Existing methodologies for patient peri-operative heating provide for interrupted heating: during “marking-up”, the pre-operative garments are removed for patient marking [as mandated in the Joint Commission National Patient Safety Goals January 1^{st} 20171, UP001.02.01] which garments may then be exchanged for intra-operative garments. Following marking up, tethered heating devices are detached from their heat source for transport to the operating room. These physiologically detrimental physical and temporal windows, typically of between 10 and 27 minutes duration, leave the patient's torso without thermal protection and therefore subject to considerable detrimental pre-operative heat loss.

In addition, when deploying existing heating methods and systems, surgical procedures conducted on the torso frequently leave only a portion of the torso actively heated, or heat the lower extremities alone, thereby reducing the efficacy of the heating device to the point where the patient's heat loss may be greater than the gain from the heating device. The instant invention, however, allows surgery to be conducted on the torso while continuous active and beneficial heating is being applied throughout the preoperative, intra-operative and postoperative phases, which heating is applied, most significantly, without interruption.

The inventors maintain that by clothing the limbs' extremities in un-tethered active thermal devices in the pre-surgical area, that vasoconstriction is reduced or eliminated, peripheral blood flow maintained and that there is, significantly, no interruption to the provision of heat to the patient, either during the marking up procedure, in transit to the surgical room, or for the duration of the surgical procedure. As a result, cardiac output, respiratory rate, heart rate, oxygenation and levels of circulating insulin, cortisol, adrenaline and inflammatory enzymes such as cytokines, are thereby advantageously maintained within the normal range.

This invention also compares favorably economically with current methodologies where the heating garments may be changed as many as four times throughout the peri-operative period. The inventors deduce that despite their intended thermal contribution, existing systems and methods for maintaining normothermia apply heat to the patient intermittently thereby allowing the onset of hypothermia. This results in detrimental reduction to core and peripheral temperature levels which the body has difficulty reversing. The inventors therefore conclude that existing systems are economically wasteful in the manner of their deployment and propose a method and system which does not require inter-operative intervention, nor result in unnecessary wastage.

The inventors assert that the instant method, system and apparatus prevents hypothermia which is of greater benefit to the patient than prior art in the field of surgical heating, which has focused on the treatment of hypothermia through the provision of heat to the patient via the torso. This, conventional approach to heating is, on occasion, supplemented by the provision of heat to the lower extremities during surgery. The logic supporting this method appears to be founded upon existing practices for the treatment of hypothermia.

Typically, for outpatients, the treatment of hypothermia is a matter of emergency treatment. The symptoms of hypothermia are proportional to the degree of body cooling and vary between mildly hypothermic; shivering, lethargy, ‘clammy’ skin and hyperventilation and the severely hypothermic state, where symptoms may include unconsciousness, dilated pupils, decreased respiratory rate and ultimately cardiac dysrhythmias and asystole. As body heat has already been removed from the extremities and the locus of remaining body heat is in the torso, the treatment for hypothermia consists of two stages; arresting the hypothermic condition by increasing the core temperature of the body, then, once the core temperature is restored, allowing the body itself to complete the reversal of hypothermia by self-regulation of temperature to the extremities. Custom and practice therefore show that the correct procedure for the emergency treatment of hypothermia is the application of heat to the torso.

The inventors contend that correcting for hypothermia and maintaining normothermia present different practical anatomical challenges and thus propose, through their invention, an alternative method and system of heating the patient for the maintenance of the normothermic condition.

The inventors further conclude that existing methods, systems and apparatus conceived for the prevention of surgically induced hypothermia have been constructed following the rationale used for the treatment for existing hypothermia, in that prior art in this field largely concentrates on heating the torso. The inventors further consider and will show that heating the extremities is a more practical solution for the prevention of hypothermia.

Existing devices for the correction of surgical hypothermia can largely be categorized into those applying heat directly to the patient and environmental thermal control devices seeking to control the patient's environment, such as heated under-blankets or operating tables which have integrated heat sources. Without exception, existing peri-operative patient thermal systems and devices require intervention, fitting, adjustment and in some cases, calibration in the operating room. The inventors perceive that inadvertent harm may befall the patient as a result of the protracted delay in applying heating. In addition, the requirement to fit existing devices in situ detracts from the workflow of the operating room. It is the inventors' intention to address this defect.

As previously noted, of the existing systems and apparatus which apply heat directly, the majority are tethered devices which convey heat from an external device to the patient via an umbilical. The inventors observe that tethered devices contribute significantly to increasing the complexity of the operating room. Additionally, in a study reported in the Canadian Journal of Surgery 53(3): 133-165, June 2010, by Wong et al report that the most common cause of delay in the operating room was attributable to equipment failure. Although clearly there are many types of surgical equipment, the inventors consider that by removing electro-mechanical heating devices, their invention contributes to making the operating room a less cluttered, safer, simpler, less failure prone environment and therefore a more effective and productive workplace.

Considered on a strictly statistical basis, it would appear that the majority of the existing intellectual property in this domain pertains to “Forced Air Warming” or “FAW” devices. There are some inventions, however, which are stand-alone in that the heat source is contained within the garment or blanket. The form of the direct heat application devices also varies widely from blankets to garments which are loosely configured about the torso.

Specifically, prior art does not, encompass the advantageous heating mode of uniquely heating the patient via the extremities utilizing single usage disposable garments. On the rare occasions where heat is applied to the extremities it is via umbilically tethered, technician intensive machines. Prior art in surgical garment apparatus should therefore be further sub-categorized into whole body garments covering the torso and garments applied to the extremities.

Prior art also includes a wide variety of different heating modes. These include electric heating elements, forced air warming devices, heat packs, fluid circulation, chemical heating and the like. The subsequent discussion concerning prior art further serves to confirm the significant advantages of the instant invention.

Anderson et al, U.S. Pat. No. 7,914,566 “Multifunction warming device with provision for warming hands” acknowledge the importance of keeping the patient's hands warm, disclosing a convective warming blanket formed as a garment with slits in the sides through which the patient's hands may be warmed by the operation of a forced air warming convective apparatus. This patent represents an improvement over Anderson et al.'s prior expansive intellectual property which is disclosed therein and which includes, among others, U.S. Pat. No. 7,364,324 Multifunction warming device with provision for warming hands. Anderson's device, while apparently increasing the versatility of forced air warming devices may also unfortunately impede patient monitoring as pulse oximetry devices typically require access to the fingers, which access may be impeded by the patient's hands being enclosed within the inflated garment. The instant invention presents no impediment to making pulse oximetry measurement.

The potential limitations to deploying partial torso heating via FAW devices in the operating theatre are acknowledged in Anderson et al.'s later evolutionary intellectual property, U.S. Pat. No. 8,097,031 Warming device with provisions for deploying elements of an upper body convective apparatus and for deploying the lower portion of the warming device, which acknowledges the importance of being able to heat a selected portion of the patient's torso, the heated portion being complimentary to the site of surgical intervention. Notwithstanding, these devices are forced-air warming devices and can therefore only heat the patient by convection: the instant device, in contrast, uses conduction and radiation as a preferred and more effective means of heating the patient.

Recent intellectual property to Panser et al, U.S. Pat. No. 8,070,797 and of common ownership with Anderson, discloses a refined clinical garment which applies heat via a forced air warming device [“FAW”] which is partitioned such that different heat modes may be applied to different parts of the body. Unlike the Anderson intellectual property, U.S. Pat. No. 7,364,324, U.S. Pat. No. 7,914,566 and U.S. Pat. No. 8,097,031 the modular construction of the Panser garment allows for sleeves to be attached to the garment in order to apply heat to the arms. Collectively the Anderson and Panser garments seek to create environmental partitions enabling differentiation between comfort and therapeutic warming. Although this increases the versatility of the heat emitter, it requires a dual mode forced air warming device machine which is attached via bulky umbilicals and which significantly leaves more of the body surface area, (BSA), unheated than is heated. Additionally, as previously noted, such devices create significant undesirable environmental impact in the operating theatre. The current invention, by applying heat directly to the patient's hands and feet is focusing the heat where it is needed, while simultaneously leaving the surgical field unencumbered and the temperature of the operating room unaltered.

The inventors note that existing surgical, patient and environmental control devices may also have undesirable bi-products in the form of increasing the heat and noise of the operating room environment. Noise levels created by the hot air blower are a distraction to the surgical team and frequently result in requests to disconnect the power in procedures of short duration or in ones in which only a small portion of the patient's body surface area (BSA) is exposed, e.g. facial, foot or hand surgery. This also reflects poorly on the efficacy and cost effectiveness of these devices. The instant device is silent, unobtrusive and does not contribute significantly to the ambient air temperature.

In addition, Albrecht et al, reporting in the American Journal of Infection Control 26(4), 317-323, in May, 2016 concluded that incorrect selection or inadequate installation procedures on the air filters used on Forced Air Warming devices can inadvertently lead to increased levels of airborne microbial contaminants. They concluded that there was little evidence that the efficiency of the air intake filter was adequate to protect the internal air path from buildup of microbial contamination within the machinery of the FAW. This, then, requires the FAW to be serviced by a technician, thereby further adding to the cost and service personnel requirements of the operating room. The instant invention has no such requirement, thus representing a significant simplification over prior art.

Forced Air Warming, “FAW”, devices, due to their inflatable nature, are inherently mechanically unstable, so that a cotton blanket is almost invariably applied over the device. Perversely, the blanket is maintained in a warming closet and also requires laundering and recycling, thereby further adding to procedural costs

In the case of FAW devices, the umbilical heating tube from the FAW device to the inflatable garment is hidden beneath its retaining blanket and surgical drapes and cannot be monitored in order to verify the effectiveness of the distal connection to the patient's garment. Nor, when accidental disconnection of the FAW occurs, is there any alarm to alert the operating personnel in the event of disconnection. The unmonitored loss of effective warming is therefore a further drawback of existing FAW devices. The instant invention has no such drawback.

Paolini et al, U.S. 2006/0132085, “Inflatable Blanket with a tie” propose a refinement over existing intellectual property in that the forced air warming blanket is equipped with integral tie-down straps. The straps serve a dual function, namely the control of the blanket position and also the degree of inflation of the device on the patient's torso. Paolini et al thereby confirm the problematic nature of securing an inflatable device to a patient in such a way as to be secure during surgical interventions. The instant invention dispenses with tie-downs.

Augustine et al, U.S. 2008/0103567, U.S. Pat. No. 8,062,329, “Heating blanket”, disclosing yet another variation on forced air warming blankets acknowledges the advantages of warming the patient's “head and at least one arm, allowing the patient's chest and abdomen [to] remain substantially exposed.” The patent states, “arms tend to be excellent heat exchange surfaces”. A further benefit claimed by Augustine is securing the heating blanket to the patient such that the blanket remains stationery relative to the patient's body position. The instant invention concurs with Augustine on the subject of heat transfer in the limbs and claims the advantage of simplicity over Augustine of being a garment, which is advantageous in that being a garment it does not require attachment to the patient and furthermore, is incapable of slippage or displacement into the sterile surgical field.

The instant invention therefore represents advances over prior art to Anderson, Augustine, Panser, and Paolini et al in that it creates a normo-thermative environment for the patient without creating detrimental heat and noise in the surgical environment. Significantly it has a lower capital cost and is preferably disposable, although a re-usable version is equally within the scope of the instant invention. Additionally, it does not require a static, multi-patient, heat device which requires constant servicing and electrical certification by trained biomedical engineers, which further increases the per-case cost as well as fixed facility overhead. Unlike blanket devices, it does not require attaching to the patient during surgery either by tethering or by a heat transfer umbilical as it is in self-contained garment form. Furthermore it is worn for the duration of the entire surgical encounter comprising the pre-surgical, surgical and post-surgical phases, which results in minimal temperature fluctuations and minimal pre-surgical thermal loss to the patient. In addition it applies heat to the limbs which is advantageous to the circulatory system. Beneficially, It may be applied in all surgical interventions where normothermia is desired and may be supplemented by passive heating means in the form of other surgical garments which may take the form of sleeves, trousers or a shirts or gowns which additional garments may be selected on the basis of their insulative properties and which are formulated on a case-by-case basis being configured so as to not impede or infringe on the surgical field.

Furthermore, the inventors believe that the simplicity of the garments is such that the garments may also be self-applied by the patient or care-giver in the hospital room or pre-surgical area and may even be applied at home prior to hospital admission.

Another embodiment of a heating device patented by Vergona et al US 2009/0299442 Warming blankets covers, and apparatus, and methods of fabricating and using the same, discloses warming blankets with pockets mounted therein, wherein each of the one or more pockets is adapted to hold a respective removable warming element. Vergona's patent specification affirms that the first two hours of surgery result in the highest degree of thermal loss per hour and proposes that the blanket will start to generate heat within 20 minutes of exposing the heat packs to the air, and continue to generate heat for approximately 12 hours which is within the time taken for the majority of surgeries.

Personal observation by Kirwan further narrows the onset of hypothermia to the first 10-12 minutes of the procedure. In this time interval, the patient is completely disrobed for purposes of marking, to apply Intermittent Compression Stockings, urinary catheters, FAW devices and to prepare and drape the patient, prior to making the initial incision. The interval between when the patient enters the operating room and first incision is always recorded and can vary from 10-30 minutes, at the end of which the patient's core temperature is typically 35° C. Partial restoration of the core temperature over time is possible using FAW devices, but the hypothermia largely remains uncorrected for the duration of the surgery.

Vergona's device further proposes that it is maintained in place purely by its own weight. The removable warming elements by which Vergona proposes heating the patient, consist of chemical compositions which, when activated by exposure to air have an exothermic reaction. This device has some advantageous design elements, in particular its lack of requirement to be attached by an umbilical, but, being a blanket it still has the potential to impinge on or contaminate the sterile surgical field and unlike the instant invention, has limited practical application in the pre-surgical, ambulatory, holding area.

The invention exposed by Vergona et al does not propose zonal focusing of the heat requirement. It is the intention of the instant invention to apply heat in a focused manner, preferentially in zonal areas of the patient's body where maximal heat absorption is assured.

Harris et al, Adjustable Disposable Surgical Thermal Blanket, U. S. 2006/0148714 teach a layered blanket containing a plurality of discrete, sealed compartments containing solid, particulate oxygen activated exothermic composition with seals defining the compartments. Harris claims advantage over Vergona in that it is disposable, additionally requiring minimal surgical staff intervention.

The invention proposed by Harris anticipates the requirement for access to the patient during surgery by permitting partial or complete detachment of one or more of the compartments to allow reconfiguration of the heating device in accordance with the particular surgical requirements pertaining to a specific operation. Additionally Harris claims pre-operative, intra-operative or post-operative blanket configuration. However, this device, as with all of the blanket devices, has placement issues, it being difficult to control positionally during surgery. Additionally, having numerous sealed pockets requires the activated pockets to be accurately placed with respect to the surgical area. Economically, the blanket is unnecessarily wasteful in that the detachable areas are disposed of without being used, thereby increasing total unit cost, yet without securing additional benefit.

The instant invention represents improvement over Harris in that it is worn by the patient, rather than being in blanket form. Additionally it targets the most advantageous zones through which it introduces heat to the patient's circulatory system, thereby requiring fewer active heating elements with which to achieve a superior heating result. This has the added economic benefit of simultaneously improving heat distribution while reducing net unit costs. It also approaches the problem of hypothermia in a more logical, physiological, way by maintaining normothermia thereby avoiding the onset of hypothermia at any time.

Furthermore the invention claims advantage over prior art through direct application of heat to the extremities of the limbs, which application is more effective than applying heat to the torso, particularly if the patient is obese.

In addition, the method proposes that applying heat to the limbs is more efficacious due to the application of heat to tissues which have superior heat absorptive qualities and distributive mechanisms. These are called arteriovenous anastomoses, “AVA's”, and are located in the extremities of mammalian limbs.

A second group of prior-art patents proposes preferential heating of the extremities. These patents can be conveniently divided into two classes: devices for surgical use and gloves for sports, leisure and work-use.

In addition to the principal requirement, which is the delivery of a measurable and stable heat supply for a protracted period of time without the requirement for intervention, further desirable characteristics are that the device is low cost, easily fabricated, and—paying particular attention to the specific requirements of the surgical environment—disposable and preferably recyclable. The instant invention fulfills all these criteria.

Walasek et al, U.S. Pat. No. 5,050,336 “Reusable and microwaveable hot or cold therapy mitt and method of manufacture” disclose a multi-layered device which can be wrapped and tethered about the patient's extremities as required.

The instant invention claims advantage over Walasek in that it is self-sustaining as it does not require a microwave or external power source to activate it. This attribute makes the instant invention readily adaptable for field or emergency use where there are no external power supplies.

Furthermore, the instant invention is pre-formed and close fitting about the patient's extremities, which format simultaneously minimizes heat loss while creating the most favorable interface for effective heat transfer between the heat element and the patient's extremities. Where the effectiveness of heat transfer in Walasek is dependent on the skill of the person applying the heat garment to the patient, the instant device requires no external support or assistance in order to arrive at optimal heating, resulting in a standardized application of heat to the patient. Finally, the chemical heat pack means used in the instant device has a linear exothermic output, providing stable heat over a longer period of time which feature can be advantageously deployed when lengthy surgical interventions are being undertaken.

Turning to more recent prior art in the domain of surgical gloves, Salmon et al, 2003/0027783, Warming Apparatus, disclose a rigid, non-conformable re-usable apparatus designed to apply radiant heat energy to a patient's hand, which advantageously uses areas of high concentrations of arteriovenous anastomoses (“AVA”) to ensure maximal heat absorption. Salmon also claims the advantage of included pulse-oximetry sensors; however, Salmon fails to address the issue of intravenous (“IV”) access for administration of intravenous fluids. In addition, detrimentally, Salmon proposes the application of heat to only one hand which the inventors assert is insufficient to combat hypothermic onset. The instant invention represents an improvement over Salmon in that it allows IV access for the administration of intravenous fluids and simultaneously provides a greater surface area for the application of heat to the patient

Although Salmon proposed the application of heat to only a single limb, requiring the limb enclosed by the active heating device to be tethered to a base unit via an umbilical. However, if the patient requires IV cannulas, the practical inference is that the IV catheter will have to be inserted into the opposite arm which is also in use for the Blood Pressure Cuff, thus leaving the lower extremity for “IV” access. This therefore requires the tethered attachment of both of the patient's arms and a lower extremity to external loci, which has significant detrimental impact on the surgical environment. The instant device has no such detrimental impact on the surgical environment.

The instant inventors consider that applying heat to the AVA's of a single limb is insufficient for the maintenance of normothermacy and that, subject to the locus of the operation, it is preferable to apply heat to the extremities of all four limbs in order to maintain thermal equilibrium. Furthermore, by maintaining the heating devices as self-contained, stand-alone units the benefits accrue to both the patient and the surgical environment.

Practically, the instant invention claims advantage in that it allows for application of a cannula to the dorsum of either hand which is being heated and equally, an independent pulse oximeter to the index finger or the big toe, thus allowing greater spatial flexibility and versatility in the surgical environment.

The instant invention also claims additional advantage over Salmon in economic terms by not requiring technician intervention and by preferentially being constructed for single use and therefore disposable.

Alternate configurations of the instant invention may be constructed to have the single-use heat elements disposed in pockets on a -reusable garment. After removal of the spent heat elements and appropriate sterilization of the garment, the pockets can be repopulated with fresh heat elements for re-use. Such alternate configurations may be considered for use in territories where obtaining surgical supplies is problematic such as field hospitals or remote locations.

In yet another variation, the chemical heat packs can be constructed in an elongated form, preferentially with heat nodules connected by thermally conductive strips, such that the heat elements and heat dispersive elements are fabricated and applied to either one of the inner or the outer surface of the garment as a single unit, thereby simplifying the construction of the device, reducing the component count and simplifying assembly.

Christensen et al, in a patent entitled Methods and Apparatus for Enhancing Vascular Access in an Appendage to enhance Therapeutic and Interventional Procedures disclose a tethered device “for increasing blood flow, controlling the vasodilatation of a patient's vascular structure, regulating the temperature of a portion of a mammal, and for improving various interventional procedures and/or therapeutic techniques.” The invention is, in effect, an electro mechanical heater, umbilically coupled to a sleeve into which the patient's limb is inserted. The sleeve controls body temperature by circulating warm water there through and also incorporates temperature measurement and feedback mechanisms and which device may also preferentially exert vacuum pressure to further improve circulation.

As with Christensen, the instant invention preferentially utilizes one or more of the patient's limbs as a means of transferring heat to the patient, but without the disadvantages of having an external power device, or of requiring significant surgical staff intervention, or of being attached by an umbilical to the patient which attachment is both inconvenient and, as previously disclosed, encroaches on the surgical environment. The instant device therefore claims benefit of simplicity over Christensen.

There are a number of non-surgically oriented patents which have as their subject matter the application of heat to the limbs and in particular the hands. These devices are typically for use by sportspeople or people who are exposed to the outdoor environment, although some devices incorporate structural support mechanisms for therapeutic usage. The majority of these inventions place the active heat element on the dorsum area of the hand such that manual dexterity and tactile sensitivity is retained by the wearer.

Early inventions focused on self-contained garment structures, whereas, more recent inventions in this class include heated gloves which derive power from external sources, typically via hard-wired umbilical connections. The advantageous nature of being able to provide heat via surgical heat garments which are not connected by umbilicals to external power means is largely self-evident. Therefore, prior art comprising tethered, heated gloves will be given only cursory examination.

An invention to Helenick, U.S. Pat. No. 6,128,801 details a combined therapeutic heat and support glove which heats the dorsum of the hand. Helenick also discloses multi-layered gloves which are fabricated to combat cold temperatures and which are designed for outdoor activities.

Helenick locates the heat element on the dorsum of the hand in order that the wearer can maintain dexterity. In the instant invention,—where the wearer has no need for dexterity, the heat element is placed adjacent to the palm of the hand. Locating the heat element on the palm of the hand, as opposed to the dorsum, makes for the greatest thermal effect, as the heat element is proximate the area of arteriovenous anastomoses which are located on the palm of the hand and which more effectively and efficiently convey heat to the patient. Furthermore, by leaving the entire area of the dorsum unencumbered, the instant device advantageously allows this area to be accessed by the surgical team for the insertion of cannulas. Practically, this area may be equipped with a re-sealable flap.

Rinehart, U.S. Pat. No. 5,035,003 teaches the use of a glove with a bladder and an external, self contained heat source, the objective being to create a convective circulatory system within the liquid filled bladder from which to transfer heat to the fingers. The instant invention claims advantage over Rinehart by virtue of simplicity in that there is no liquid filled bladder and also by economic advantage of simplicity of construction and disposability which is advantageous in the surgical environment. Furthermore, as previously mentioned, the heat distribution from the chemical heat means proposed in the instant invention represents an improvement in that the heat output is both linear and more controllable than heat dissipated from physical heat elements, which suffer from thermal decay.

In a more recently awarded patent, U.S. Pat. No. 7,029,768 to Gogarty, reveals a thermal gel filled glove. The glove is heated and largely disposed about the wearer's hand. Although designed for therapeutic treatment, the device would prove problematic in the surgical environment as the gel filled elements preclude intra-venous catheterization. The instant invention claims the advantage of simplicity, improved economics and utility over Gogarty as the heat transfer element is integral to the glove, which heat dissipation mechanism allows for heat transference across a larger body surface area, while simultaneously allowing for cannulization of the patient.

Haensel, U.S. Pat. No. 6,218,335 Circulation Warmer, proposes an apparatus and method for warming the blood of a user prior to distribution to an extremity of a user which includes a source of energy suitable for distribution as heat. Haensel's device incorporates transfer paths, heat positioners and energy state changes, which represent a considerable degree of complexity. Such complexity is inconsistent with the requirements placed upon a normothermic device by the demands of the surgical environment and therefore, the instant invention claims the benefit of simplicity over Haensel as the device requires no integral energy state changes and additionally there are no fluid transfer paths incorporated therein.

Finally, the construction of the instant device represents a simplification over prior art in that, with the exception of a pocket which contains the heat element on the palm area of the garment, the garment construction preferentially comprises a single fabric layer. This construction method reduces costs, improves economic viability and represents a level of simplicity not envisaged by prior art.

### SUMMARY OF THE INVENTION

The object of the invention is the maintenance of normothermia in patients undergoing surgery. The distinction between prior art which sought to restore the patient's hypothermic condition and the instant device which seeks the prevention of hypothermia in the patient, is important as it confers numerous benefits on the patient.

Whereas prior art typically utilizes tethered, bulky, heat transfer mechanisms attached by umbilical to technician intensive electro-mechanical support machinery and requiring intra surgical calibration by surgical staff, the current invention, requiring none of these, represents a considerable simplification over prior art as deployed in the surgical environment.

In practice, ideally, the patient clothes themselves in the device as instructed by surgical staff and the clothing is then worn for the duration of the surgical procedure. The garment requires little or no technical intervention by surgical staff. In addition it may be equipped with simple visual thermal indicator means which diagnostic reports on the device functionality and which assists surgical staff with monitoring patient welfare.

Unlike prior art which subordinates the needs of the both surgical staff and surgical environment to the patient's requirements, a further consideration of the instant device is that it enhances utility from both the patient's and the surgical team's perspective.

In describing the basic structure of the invention the words “glove” or “garment” will be used. This is not intended to provide constraints on the invention. Persons skilled in the art will clearly understand that the device is intended to provide heat to the extremities of mammalian limbs, thus, using “glove” as a descriptive term is not intended to preclude the use of mittens, bootees, socks, slipper-socks or similar garment forms. Nor is the descriptive term “glove” intended to provide any dimensional constraints, such as, to the length of the cuff, which may be of any practicable length, as desired. In a similar manner the description is not intended to provide a limiting basis for the dimensional construction of the device for use on the lower limb. Furthermore, it should be noted that there is advantage in forming the glove into a mitten as it results in a lower surface area, which reduces heat-loss, which is an important goal of the invention. Accordingly, FIGS. 3a, 3b and 4b illustrate an embodiment of the invention configured as a mitten.

In further describing the invention, for the sake of simplicity, the singular will be used. This is not intended to infer a limitation to the device, but rather to indicate that the device may be deployed either on a single limb or on multiple limbs, depending on both the individual patient's thermal requirements, the proposed location and duration of the surgical intervention and the ambient temperature of the operating environment.

As previously disclosed, the instant invention pertains to the method of maintaining normative body temperature in surgical patients in the peri-surgical environment utilizing a self-contained, untethered, heat supply garment. This method should be favorably compared and contrasted with the existing surgical methodology which allows the patient to become hypothermic prior to applying remedial means in order to restore normothermacy

The device takes the form of a passively insulative garment which clothes or envelops an extremity of a patients' limb, to which at least one integral active heat-element is applied. The garment, therefore, clothes the patient's extremities and is worn by the patient throughout all phases of the surgical encounter. The patient thereby benefits peri-surgically from an uninterrupted application of heat with which to assist in the maintenance of normothermic body temperature.

A key feature of the invention is that it requires minimal or no technician intervention, thereby accomplishing the goal of maintaining patient normothermia while simplifying the surgical environment.

The instant garment is worn such that it beneficially targets areas of the patient's physiology where there is an inherently high thermal transfer coefficient, thereby maximizing conductive heat transfer from the device to the patient. These areas are typically located at the extremities of the limbs. Once clothed in the garment heat-elements are activated, providing up to 12 hours of heat to the patient, which time period is sufficient to accommodate the peri-operative duration of the majority of surgical interventions.

A further feature of the garment is that it is constructed such that there is minimal heat loss to the environment. This is beneficial in two ways: it provides for effective heat economics within the device while maintaining the ambient temperature for the further benefit of the surgical staff.

Heat is dissipated within the garment either by contact, via direct conductive means, or via a tracery or weblike network of heat-distributive elements. Alternatively, the heat may be transferred from the heat-element to the patient convectively via optional profiled heat channels located close to the patient's skin. Modification to the shape and surface area of the heat pack elements themselves is also considered to be within the scope of the invention. This would enable the heat packs to be sized according to the patient's specific heat requirements and also allow them to be fitted linearly in proximity to the patient's veins and arteries.

The heat elements are preferably not in direct contact with the patient's skin Heat loss to the environment, in the area proximate the chemical heat element, is minimized through provision of a simple, insulated, ventilated, external cover. Indicators of the garment's performance may be advantageous with preference being given to low cost, sensor free indicators. In one preferred embodiment, thermal inks may be marked onto the external surface of the garment and via heat conductive elements, directly connected to the area of the garment proximate the patient's skin. These thermal indicators, or similar devices, may be used to provide surgical staff with simple visual confirmation of garment functionality without introducing unnecessary complexity to the invention. A further economic benefit of this selected diagnostic feature is its simplicity and cost effectiveness, in that it does not require external power sources or expensive instrumentation and therefore lends itself to post-operative disposability or recycling.

A further physiological benefit, derived from the maintenance of normothermic temperature, is the avoidance of peripheral vasoconstriction. Maintenance of normal vascular perfusion maximizes the diameter of the veins which greatly facilitates the insertion of an IV cannula in the pre-operative and intra-operative phase as well as aiding the monitoring of arterial hemoglobin oxygen saturation via pulse oximetery during surgery. Therefore the instant invention suggests an additional practical advantage of applying heat mechanisms to the limbs as it offers greater opportunities for problem-free provision of intra-venous access, and improved patient condition monitoring which advantages are invaluable to both patient and surgical staff.

In summary, the instant method and device provides a superior method of providing heat to a surgical patient, reducing patient morbidity, while simultaneously providing for increased surgical access during surgery while minimizing undesirable spatial and thermal effects on the environment of the operating room.

### BRIEF DESCRIPTION OF THE DRAWINGS

FIG. 1: is a timeline schematic of the patient's core temperature taken in relation to peri-operative procedures, showing the benefits of the instant method when compared with existing devices and methodologies.

FIGS. 2a through 2c: show three-dimensional and partial cutaway drawings of the instant device configured as a glove. FIG. 2a is a three-dimensional and partial cutaway drawing of the dorsal view of a hand wearing the invention. FIG. 2b is a three dimensional and partial cutaway drawing of the ventral view of a hand and FIG. 2c is a lateral medial cross section of a hand, both of which are wearing the invention.

FIGS. 3a and 3b show three dimensional and partial cutaway drawings of the instant device configured as a mitten. FIG. 3a is a three-dimensional and partial cutaway drawing of the dorsal view of a hand wearing the invention. FIG. 3b is a three-dimensional and partial cutaway drawing of the ventral view of a hand wearing the invention.

FIGS. 4a and 4b show three dimensional and partial cutaway drawings of the instant device configured as with proximal extensions in form of a gauntlet.

FIG. 5a shows a detailed three dimensional semi-transparent, cutaway drawing of the beneficial features of the heat element contained within a protective cover.

FIG. 6: shows a three-dimensional and partial cutaway view of the device as configured for a lower limb, showing two configurations: FIG. 6a as an ankle sock and FIG. 6b as an elongated knee-length sock.

### DETAILED DESCRIPTION OF THE PREFERRED EMBODIMENTS

It will be readily understood by those practiced in the art that the components of the instant invention as generally described and illustrated herein can be designed and arranged in a variety of different configurations. Thus, the accompanying more detailed descriptions of the embodiments of the system and method of the present invention are in no way intended to limit the scope of the invention. Additionally, those skilled in the art will appreciate that various modifications to the detailed schematic diagrams contained herein may be made within the scope of the invention without departing from the essential characteristics of the invention as described herein.

The instant device comprises a method, system and apparatus pertaining to the deployment and disposition of a disposable or substantially recyclable heating garment preferentially configured for application to the extremities of the patient's limb or limbs.

The method of deploying the instant device commences in the pre-surgical environment, where the patient disrobes and clothes themselves with surgical garments. The instant device is then worn on, one or, preferentially, more than one limb, as indicated by surgical staff. This method and application of the invention represents a significant advantage over existing methods and technologies in that the patient's core body temperature does not have time to be deleteriously affected prior to entering the operating room. A further advantage of the instant device is that the patient dresses themselves, removing the requirement for intervention by surgical staff. This helps to preserve patient modesty and avoids any risk of liability arising from inappropriate actions on the part of the attendant staff.

Yet another method advantage is the simplification of the patient-care process: utilizing the instant device does not require sophisticated and expensive staff intervention, nor does it require that the operating room is cluttered with additional support equipment.

Therefore, in the method of deployment, at a minimum, the patient encases one extremity or limb within the confines of the device. However, dressing more than one limb is considered advantageous as it reduces the skin area of the body which is exposed to ambient room temperature while simultaneously increasing the body surface area, “BSA” benefiting from heat generation generated by the instant device.

The deployment method of the instant invention is favorably compared and contrasted with existing surgical devices which seek to insulate and heat the torso. Assuming that a particular surgical procedure allows for the anterior and posterior of the entire torso to be heated, the resultant body-surface area for applying heat and insulation is 34%. This figure assumes the application of heat to the entirety of both front and back of the torso. However, typically, the application of FAW devices is restricted to the anterior aspect of the body and, occasionally the anterior aspect of the extremities. As a consequence of this, if surgery is to be performed on the anterior torso of the patient, the area of total coverage may suffer further reduction, resulting in an active heating area of 13% or less of the BSA, while simultaneously exposing a large proportion of the BSA to the cooling effect of being uncovered.

By comparison, even if the instant device is only applied peripherally to the extremities of both lower limbs and arms, i.e. the hands, and feet, the resultant total area of the body which is heated and insulated would be ˜13% of the total body surface area. Clearly, the instant invention applies heat across a lower body surface area than existing devices; however, the reduction in BSA coverage is more than compensated for by the superior quality of heat receptiveness and heat transmission of the area being heated.

Functionally, the ability to apply heat to more receptive components of the BSA, without impinging on the site of surgery represents a significant improvement over prior art.

The inventors also perceive that their invention may be successfully and advantageously deployed in multi-site surgical interventions such as liposuction. Thus, used in conjunction with standard or specialized surgical garments, the instant invention preferentially provides a method of applying both passive insulation and active heat distribution in a manner which is least disruptive to the surgical process while simultaneously accomplishing the invention's objective of maintenance of normothermic patient temperature.

In addition to the normothermic benefits conferred to the wearer of the instant device the inventors consider that the method of deployment of their invention will serve to reduce the incidence of patient morbidity as, unlike existing devices which seek to recover the patient's core temperature, the instant method and apparatus acts to prevent the onset of hypothermia during pre-operative preparation. Patient morbidity, as previously noted, consists of a range of sequelae which may include deep vein thrombosis, hematoma, infection, and cardiac dysrhythmias and ischemia.

The inventors also consider that, in line with The Joint Commission National Patient Safety Goals, NPSG.03.05.01, January 1^{st}, 20171, the instant invention may have significant benefit in reducing the requirement for anticoagulation therapy. The Commission noted that; “Anticoagulation medications are more likely than others to cause harm due to complex dosing, insufficient monitoring, and inconsistent patient compliance.” The inventors note that the prevention of hypothermia has significant benefit in terms of improving patient circulation, preventing the formation of blood clots, particularly in the extremities and thereby reducing the requirement for anticoagulation therapy.

Notwithstanding the several benefits conferred by the instant device in active heating of the extremities, use of the instant invention does not preclude the advantageous deployment of passive heating garments to the remainder of the patient's torso.

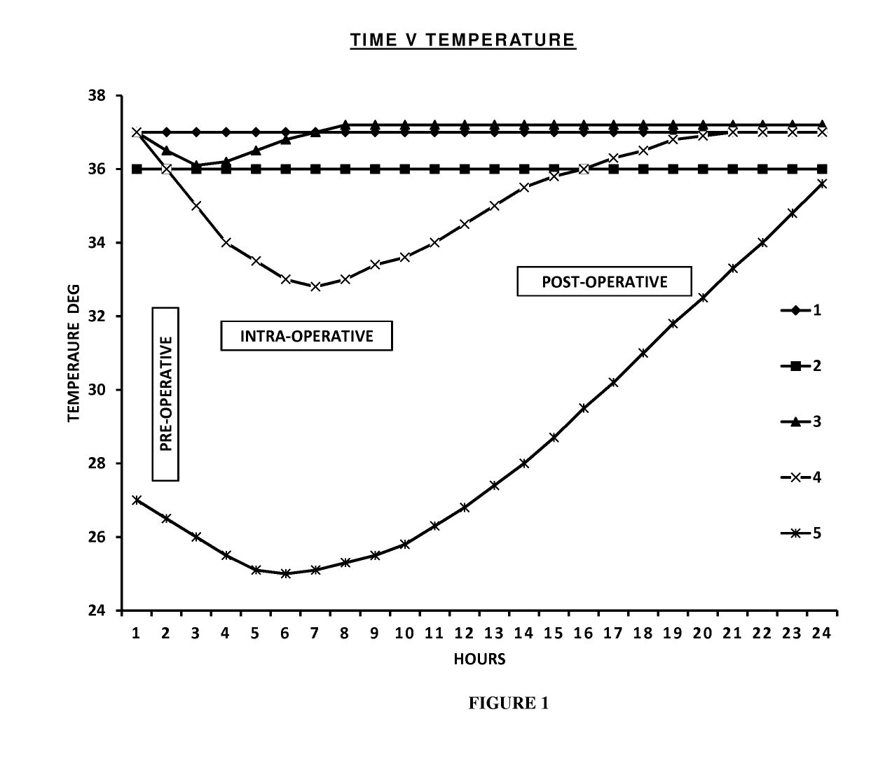

Turning now to FIG. 1 which illustrates a hypothetical timeline with relevant associated patient temperature curves and which serves to illustrate the beneficial method of the instant device. The normative baseline core temperature of a human body is represented as a horizontal line at the 25° C. meridian 1. A second line, drawn at 36° C., 2, indicates the upper temperature limit for the onset of hypothermia. It is the inventors' intention to calibrate the heat outputs of the instant invention such that an idealized desired temperature decay curve as illustrated at 3, is attained.

As previously discussed, standard pre-operative medical procedures typically result in reduction of patient core temperature. The anticipated average heat loss during this pre-surgical period is between 1.5° C. and 2.0° C., 4. Further thermal degradation of the core temperature is exhibited during the “marking-up” and surgical preparation, [M] when the patient's torso is left substantially unclothed. On initiation of the surgical procedure, the body is typically equipped with heating means, which, at best, enables partial recovery although which more typically prevents further degradation of core temperature during the surgical procedure: this is—illustrated by temperature curve 4 Note that there is a prolonged delta temperature offset between the normal and the partially recovered temperature, (x). Whereas the temperature offset between normative core temperature 1 and recovered temperature may be relatively small, the same cannot be said to be true for the offset temperature between core and the extremities, where the skin temperature may be as much as 171° C. lower than the core temperature. Clearly such a large negative temperature offset is undesirable and carries with it attendant health risks for the patient.

As an example of core temperature recovery using FAW, an initial, theoretical patient lower limb temperature, with a negative −4° C. offset and initial, ideal temperature of 25° C. has been selected 4. For a patient with impaired circulation, utilizing existing medical devices may result in a temperature decay curve to the extremities which mimic the core temperature decay, 5. Normal temperature is only restored through prolonged application of FAW or other external heat sources as the patient is incapable of restoring core temperature without assistance. The resultant delta offset prior to correction of the core temperature through provision of heat from a conventional heat source is labeled as (y).

The principal reasons that the corrective delta temperature offset never closes with baseline core temperature is the result of heat transfer inefficiencies in the method of convective heat application, the application of convective heat to poor quality insulative body tissue and the body's own biochemical response to being both hypothermic and unconscious.

By comparison, it is the intention of the existing heat method to maintain normothermic core temperature 1 and enhanced skin temperature 3, to the extremities by applying conductive (rather than convective) heat across a more receptive component of the body surface area.

Although, as a primary indicator of the stability of patient health, it is clearly important to maintain body core temperature, 1 in terms of reducing patient morbidity it may be more significant to reduce the offset between core temperature and the skin temperature of the extremities 5. The instant invention seeks to accomplish this through heating of one, or preferably more than one of the extremities.

The laws of physics relate the magnitude of heat loss to the temperature offset between two adjacent elements. Therefore, increasing the temperature of the patient's extremities as illustrated by the curve labelled 5 to mitigate heat loss from the core may ultimately prove to be as important to patient wellbeing as maintaining core normothermic temperatures 1. A typical reduction in base temperature, after the application of heat to the torso is illustrated at “y” and the proposed offset temperature range after heating the extremities is illustrated at “z”. What is evident is that the greater the temperature offset between core and limbs, the larger the potential for heat loss from the body core and the steeper the resultant patient's temperature decay curve.

Turning now to the construction of the garment 10, the specific physical requirements of the garment 10 demand the use of materials with particular properties which promote and sustain garment functionality. The glove 10 should be constructed primarily to confer thermally insulative properties. However, unlike prior art in the field, the instant invention proposes construction using a single layer of fabric 9. This represents a simplification over prior art forms which have typically used multiple fabric layers in a laminar construction in order to confer all the necessary garment attributes. Despite the advantageous economic and environmental constructional use of a single fabric layer, the inventors acknowledge that, in order to achieve optimal functionality, the glove may require the construction to incorporate fabric types with particular properties in order to optimize performance of specific facets of the garment design. For example, the thermal attributes of the device 10 may be improved through the inclusion of an elasticated cuff, 13 which demands a different type of fabric 11 from the remainder of the glove 10. Therefore, nothing in this specification precludes the incorporation of fabric types having different single-layer weights or properties being placed at strategic locations in order to achieve a desired result. For purposes of clarification, it is the inventors' intention that the only dual-layered part of the garment 10 is that which is required in order to retain and provide insulation to the heat-pack 14 which is co-located on the palm area 27 of the hand.

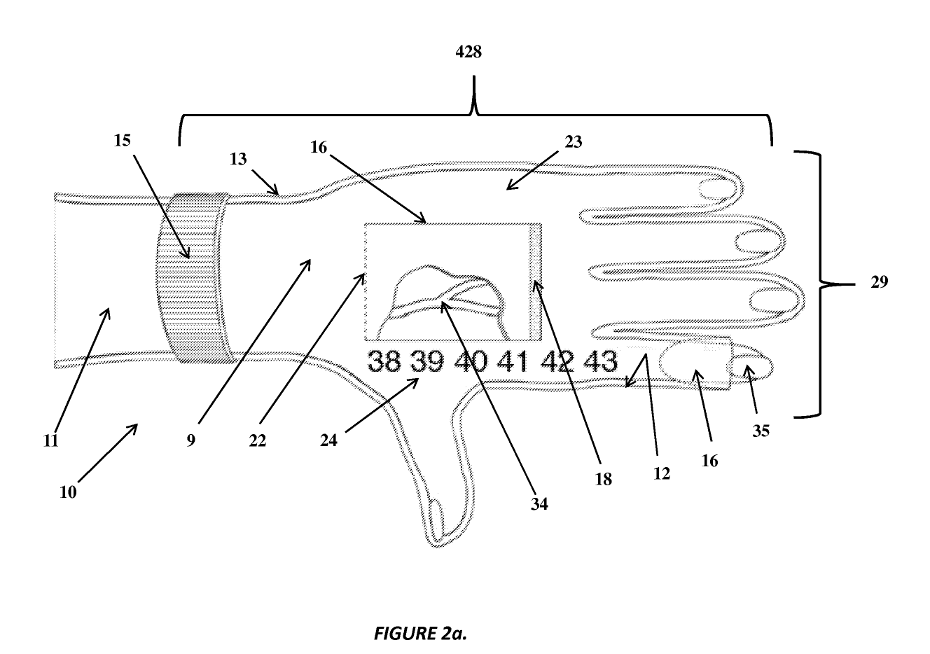

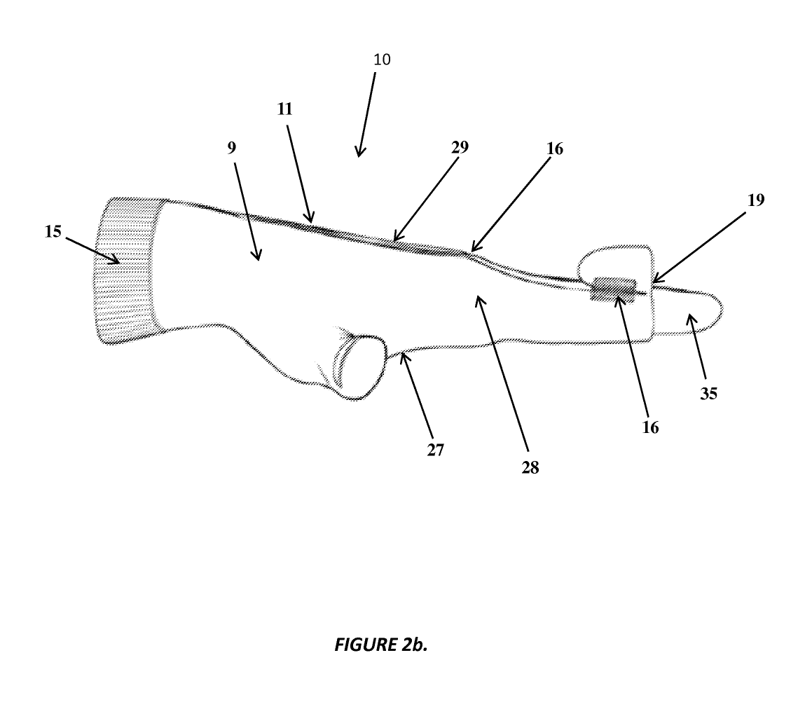

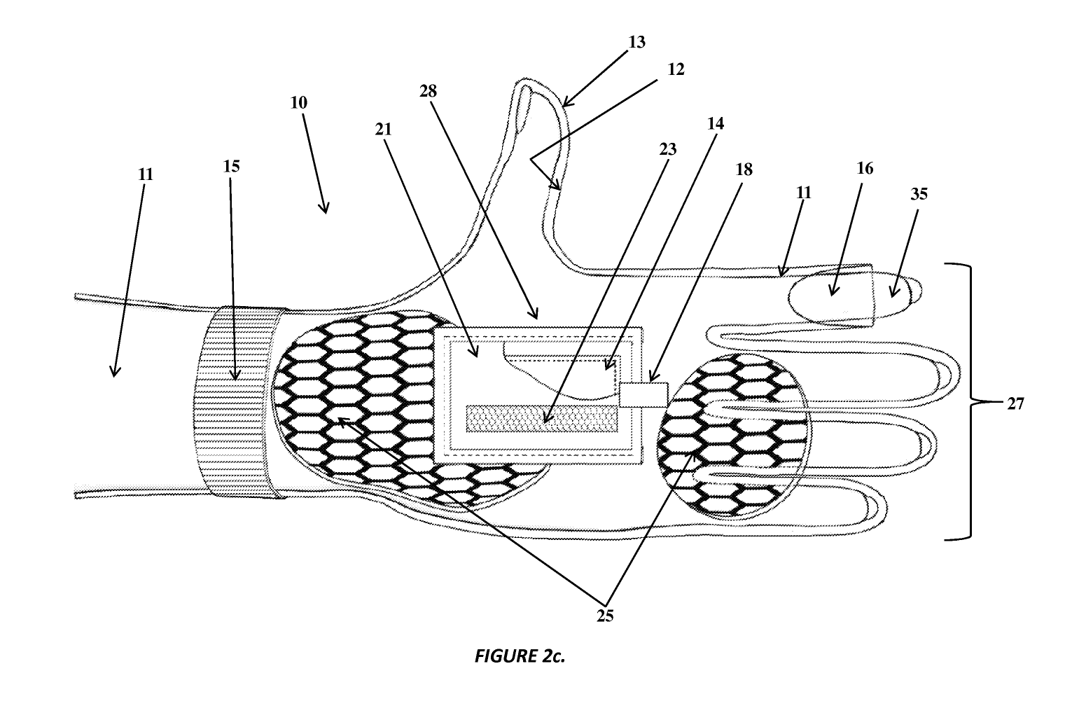

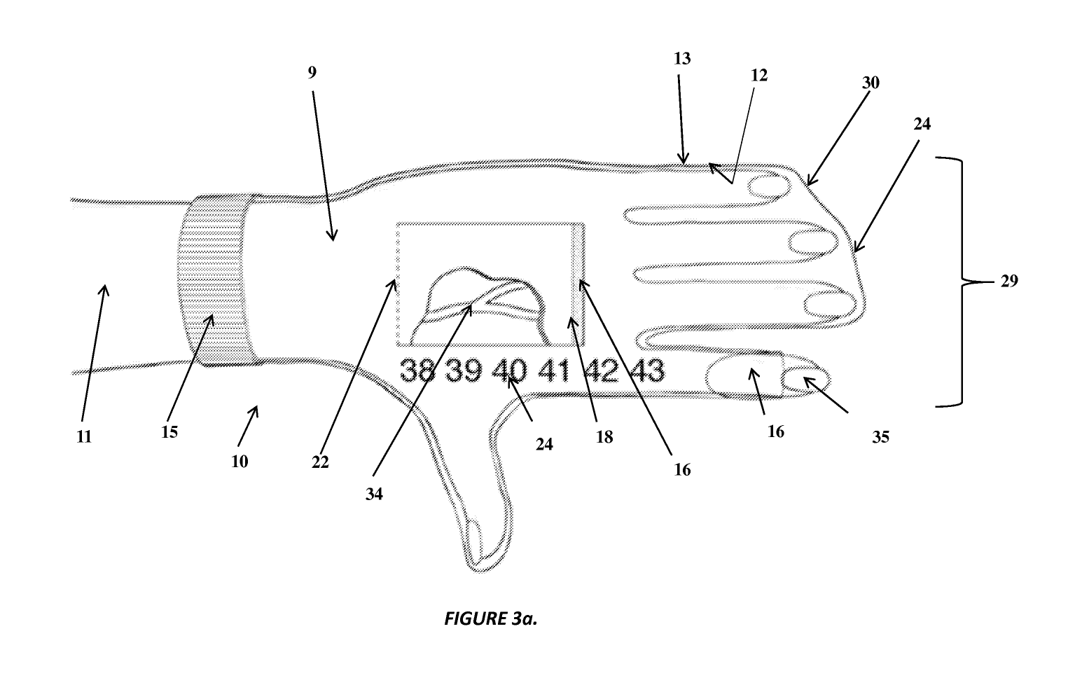

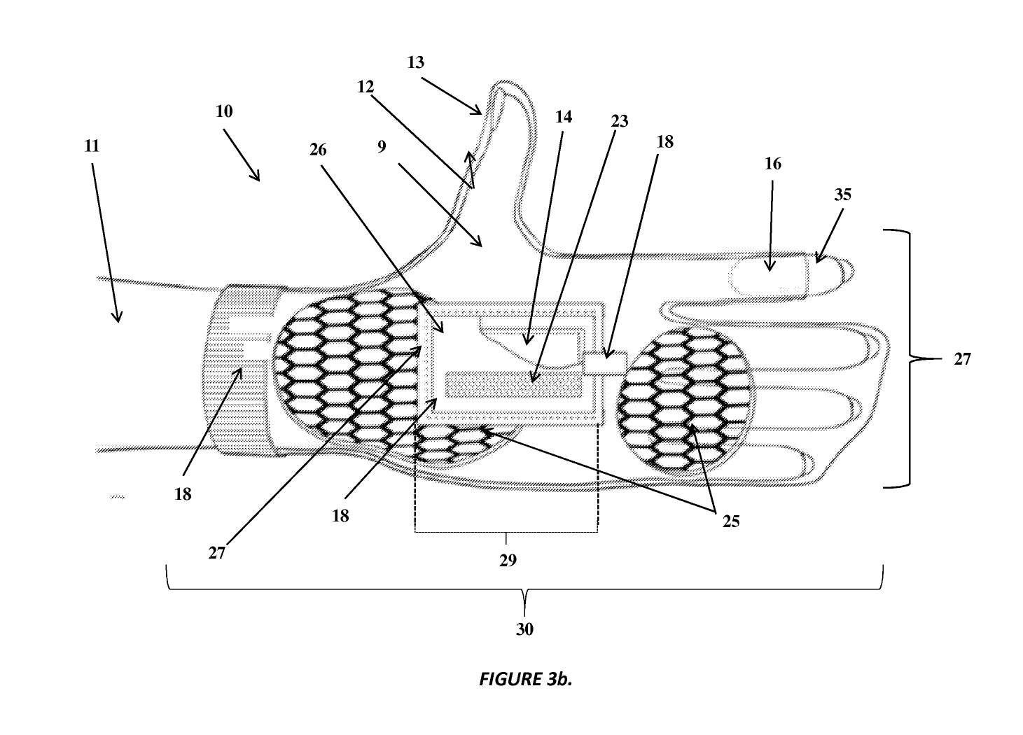

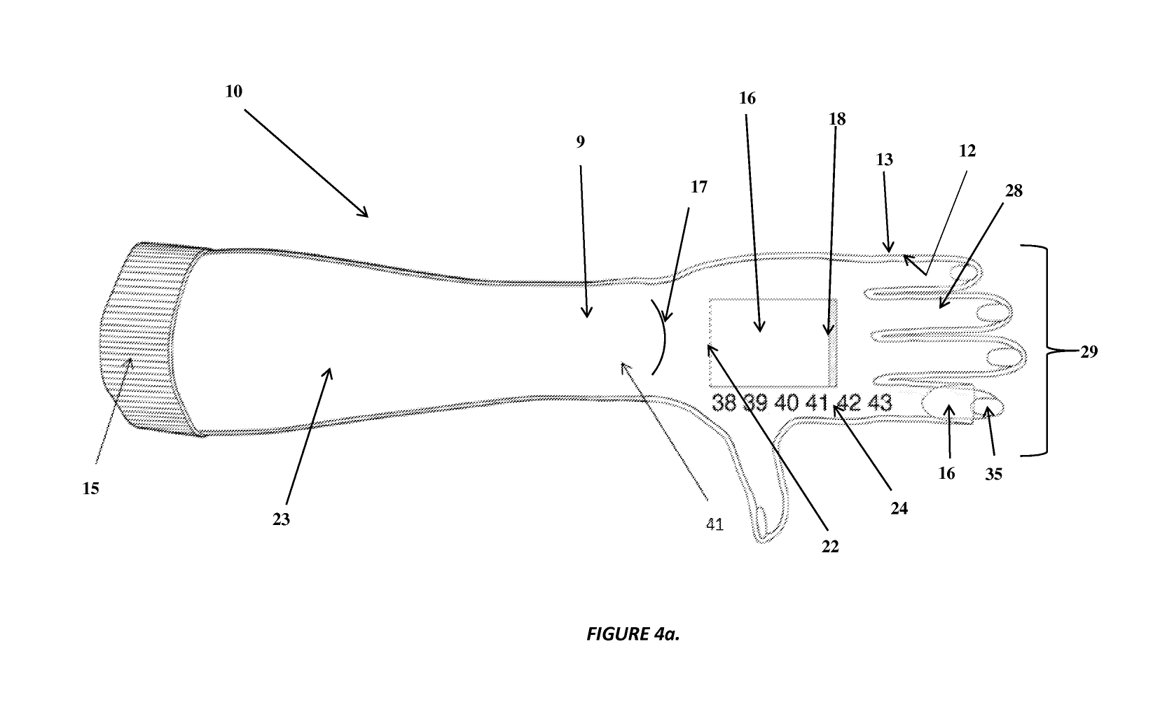

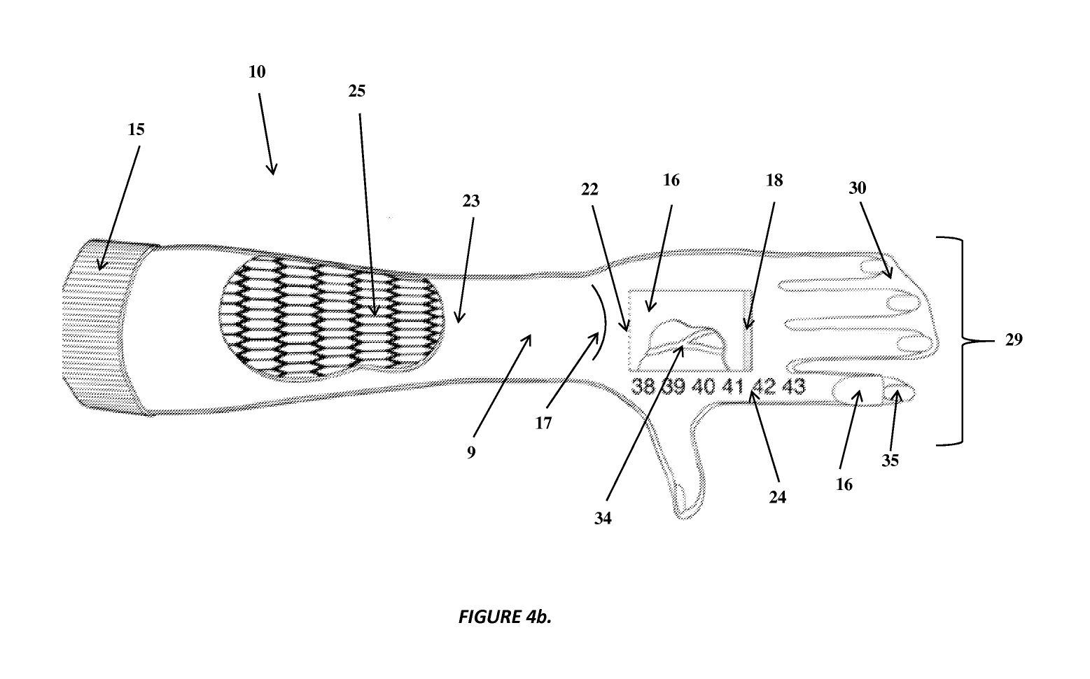

FIGS. 2a, 2b, 2c and 3a, 3b and 6a expose the embodiments of the invention which cover the smallest body surface area. The device 10 when configured for upper body usage takes the form of a glove 28, or mitten 30, or when configured for a lower limb, takes the form of a sock or boot 32. The glove 28 may have variable lengths, as illustrated in FIGS. 2 and 4 and may cover from the fingertips 52 to the axilla (not illustrated). In a similar manner the sock 32 may be extended proximally to provide coverage of a greater skin surface area. In its basic form the glove 28 has a heat pack 14 preferentially located on and integrated into the ventral structure of the glove 28 covering the palm 27 of the hand, whereas the sock has a heat pack 14 preferentially located in proximity to the sole of the foot 31.

Prior art in the field of provision of heat to the hands, when deployed in glove format, sought to preserve tactile sensitivity which precluded placement of a heat pack 14 in the palm area 27 of the glove 2816. In the surgical environment, there is no detriment to temporary removal of feeling from the palm area 27, which advantageously allows the instant invention to deploy the heat pack 14 in the palm area 27 of a glove 28.

By placing the active heat element 17 of the of the device 10 in proximity to the palm 27 to introduce heat to the body, the instant invention makes preferential use of the position of arteriovenous anastomoses located in the palm of the hand 27. Arteriovenous anastomoses, or “AVA's”, are areas of the body where a nexus of veins and arteries are proximate to the surface of the skin 171. These areas have been shown to have significant influence over body temperature—an influence which is disproportionate to their body-surface area. An experiment which confirms this attribute, entitled “Heat extraction through the palm of one hand improves aerobic exercise endurance in a hot environment”, was conducted by Dennis Grahn and Craig Heller at Stanford University and reported in the Journal of Applied Physiology (September, 2005) summarized “The heat extraction technology takes advantage of adaptations for heat transfer that are features of certain non-hairy skin surfaces. The arteriovenous Anastomoses (AVAs) and venous plexuses in the palms of the hands and the soles of the feet are effective mechanisms for heat dissipation when core body temperature rises.”

Although Grahn et al's study was used to quantify improvements to the subject's endurance as a result of direct application of cooling to the palm of the hand, it is the intention of the inventors to advantageously place the active heat element in juxtaposition with the same highly efficient zone of skin and blood vessels in order to maintain normalcy of core temperature in the patient's peri-operative condition.

For the lower limbs 20, the areas benefiting from passive insulation include the soles of the feet 31 and popliteal fossa, (not illustrated). These areas are thermally advantageous because, as previously noted, the circulatory system is in close proximity to the surface of the skin 11.

Although it is within the scope of the instant invention to have the heat-pack 14 placed adjacent the patient's skin 11, the inventors consider that this compromises the efficiency, effectiveness and versatility of the instant method and device 10 without yielding advantage. Placement of the heat pack adjacent the patient's skin 11 provides an artificial upper temperature limit on the chemical heat pack element 14 which constrains the heat element's thermal reaction and may thereby compromise heat output. If placed in direct contact with the skin, 11 the heat element 14 may produce undesirably high thermal output which may inadvertently cause the patient harm. As the preferred heating mode is derived from an air-activated exothermic chemical source 14, the exothermic reaction may be controlled by varying the amount of air which the heat element 14 absorbs. However, the inventors assert that having to regulate the air-flow to the heat element 14 in order to control thermal output, introduces unnecessary complexity to the operation of the device in addition to placing additional burden on the surgical staff. The inventors therefore determine that varying the chemical reaction speed though adjustable air intake means, although within the scope of the invention, is impractical and is not therefore a preferred embodiment of the invention.

Therefore, the inventors propose that the preferred means of controlling exothermic output is by varying the insulative and heat distributive capabilities of the device through various design features of the garment 10. The inventors therefore conclude that, although placement of the heat-element in direct juxtaposition with the patient's skin is possible, there is greater practical benefit on interposing an insulative garment layer between patient and heat element which results in intervention-less operation.

The heat element 14 is therefore situated on the garment's outer surface 13 and advantageously positioned to distribute heat to areas of the skin 11 which have greater heat absorption capability 20 as illustrated in FIGS. 2c, 3c, 4b, 6a and 6b. Advantageously, the heat pack 14 may have its interior face (not illustrated) resting on a web of thermally conductive filaments 25 so as to transfer heat from the area immediately beneath the heat pack 14 across a larger skin surface area thereby increasing the actively heated proportion of the patient's limb 19, 20.

For the upper extremities 19, as illustrated in FIG. 4, depending on the particular heat requirements of a patient, the glove elements 28 may be elongated proximally, simultaneously providing additional active heat distributive means and passive insulation to the arms 19,

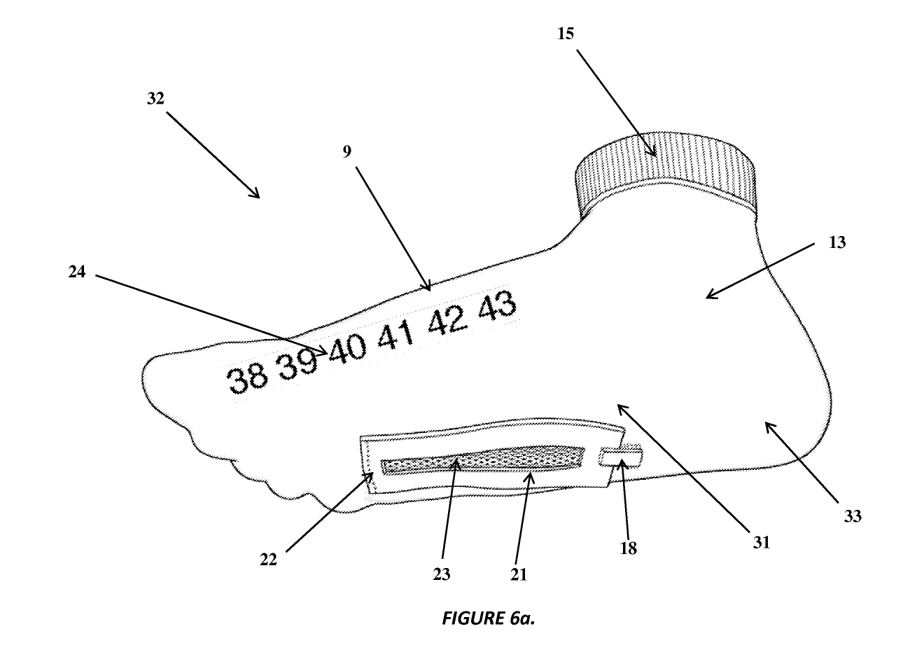

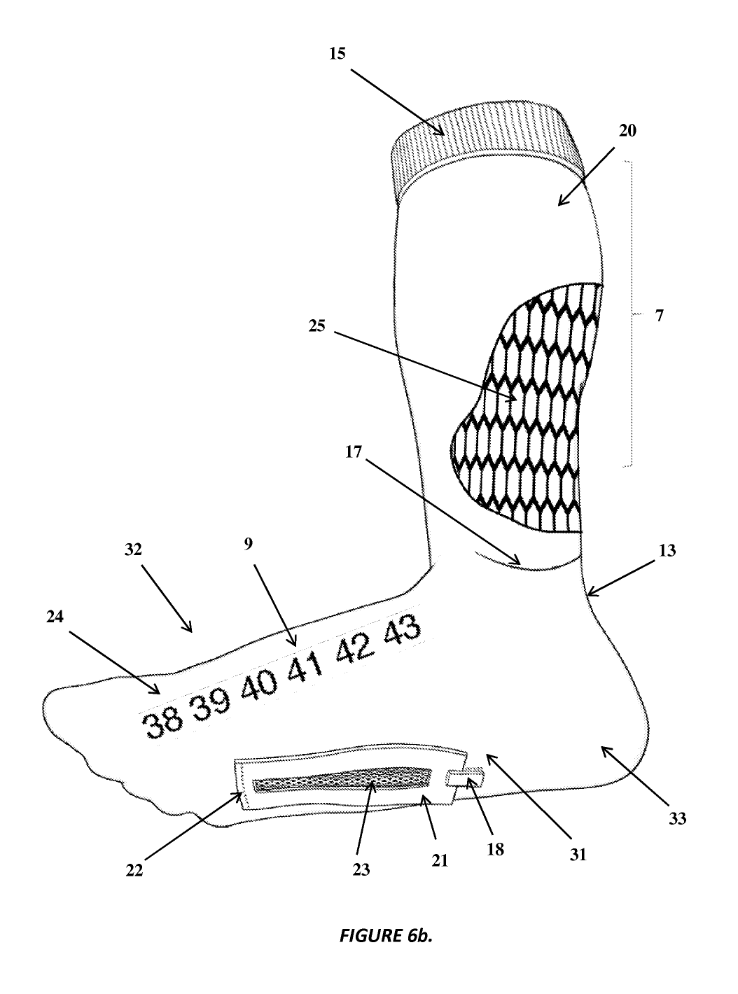

In a similar manner, as illustrated in FIG. 6b, the garment 10 may be extended along the axis of the lower limbs 20 such that the lower limb 20 may be enclosed within the garment 10 and heat applied thereto. Clearly, the invention's versatility is enhanced through coverage of any combination of limbs 19, 20, including partial or extended axial coverage of each limb 19, 20 which may be made in order to accommodate patient or specific surgical requirements without departing from the spirit of the invention.

It is preferable for the garments 10 for the lower limbs 20 to be configured to have a sock-like element 32 at the distal end. Incorporating a pre-formed heel section 33 into the device 10 enables the patient to dress themselves while ensuring that the heat pack 14 orientation is adjacent the sole of the foot 31. Alternatively, the lower limb garment 10 could be constructed as a complete “ankle” sock 32 with toes.

The plantar face 31 of the sock 32 is equipped with an exothermic heat device 14 which applies heat advantageously to the sole of the foot 31.

The glove is constructed of a single layer of fabric 9 which has an inner surface, 12 proximate the patient's skin 11 and an outer surface, 13 which is exposed to the atmosphere. The material, 9 whether paper or fabric or any other form of flexible or pliable material is selected from a group of materials which have the required structural, insulative and thermal properties. The garment 10 may be fabricated from one of a class of fabrics which are manufactured in essentially continuous reels of tube like materials or, alternatively, may be constructed from rolls of fabric, cut, stitched, glued, welded or formed in any other manner, as required, or manufactured from a combination of tube like and sheet materials, without departing from the spirit of the invention.

Notwithstanding the single-layered construction, the fabric surface 12 proximate the patient's skin 11 may contain reinforced or thickened gussets or inserts which convey particular properties at predetermined and specific parts of the garment 10, as required, in order to accomplish the goal of structural integrity, or efficient distribution and retention of heat energy to the patient's extremities. The characteristics of a material 9 are selected therefore, from a range of properties which promote comfort, passive warming, and heat dissipative and insulative properties. Furthermore, by fitting the garment 10 in relatively close proximity to the skin 11, air-flow between the patient's skin 11 and the device 10 can be controlled, thus enhancing the thermal transfer and insulative effectiveness of the device 10. For this reason the fabric surface 12 closest the skin 11 may be profiled, quilted or textured (not shown) to channel warm air in proximity to the patient's skin 11.

In a further beneficial arrangement, the fabric layer 9 which is deployed betwixt the heat pack 14 and the patient's skin 11, should have insulative properties which mitigate heat concentration and maintain the heat supply at a desired skin temperature of T<41.5 C. If temperatures in excess of 41.5° C. are applied directly to human skin over a significant time period, the skin may be damaged by excessive heat concentration. The lower threshold for heat damage to skin due to heat is reported and defined by Moritz and Henrique (1631) as either 44° C. or 45° C. More recent works, in particular that of Xu and Qian (1695) predict the time threshold for a first degree burn to be 16 hours at a skin temperature of 42° C. and 6.5 hours at a skin temperature of 43° C. The material 9 between the patient's skin 11 and the heat element, 14 should therefore incorporate thermal regulatory properties which allow the maintenance of temperature delivery to the patient within the range 40.5° C. to 41.5° C.

Therefore, the material selected for the construction of the fabric layer 9 should have thermal and insulative attributes which are proportional to the heat output of the selected exothermic device 14.

Additional features may be incorporated either within the weave of the material layer 9, or into the texture of the inner surface 12 of the material, 9 which is adjacent to the patient's skin 11 in order to provide improved heat distribution. In the event that the garment 10 is extended to substantially cover a limb, these features may preferentially take the form of thermally conductive filaments 25. The filaments 25 may be arranged to run co-axially with the principal axis of the patient's limb 19, 20, in a web like network, as illustrated, to follow the principal axes of the venous or arterial routes which have the highest effective heat transfer rate and therefore a disproportionate influence on biochemical responsiveness to heat loss. Alternatively, the heat distributive mechanism may be configured in any appropriate manner which promotes effective heat transfer from the exothermic heat element 14 to the patient's skin 11.

In addition, the heat transfer filaments 25 may be disposed about the inner surface 12 of the fabric 16 of the device 10, in any manner which is conducive to effective heat transfer; but, for preference, by adhering to the surface 12, or by weaving or embedding the heat transfer mechanism filaments 25, within the body of the fabric 9 prior to fabrication of the device 10.

The garment 10 may be equipped with a variety of openings 16, 17, which act to enhance garment utility in the surgical environment.

These openings may take the form of detachable or incomplete seams and hinged openings 16, 17 in the instant device 10. During surgical intervention, for example, it is often necessary to insert a cannula (not shown) into a vein 20. Traditionally, the point of insertion is located on the dorsum of the hand, 29. Advantageously, positioning the heat pack 14 in the palm area of the hand 27 still allows for the inclusion of one or more openings 16 in the area adjacent the dorsum of the hand 29 through which to insert a cannula (not shown). Once the cannula insertion has occurred, the opening 16 may be partially re-sealed to prevent additional unwanted heat-loss. The re-sealable opening 16 may be configured as an overlapping fabric section, a hook-and-loop attachment, as illustrated, 18 or as a Ziploc® type feature or any other re-sealable means which has the desired utility.

In an alternate configuration, the opening inset into the fabric, 16 may comprise either an incompletely stitched seam, or an opening comprising optional and selectable single-use perforations which are not re-attachable nor re-sealable. In alternate embodiments, illustrated in FIGS. 4a and 4b, and 6b, where the garment 10 is axially extended to cover the forearm, 19 or calf, 20, the openings 16 may take the form of a circumferential cut 17 around the wrist or ankle allowing access to points of specific diagnostic and surgical interest, whereby instrumentation may be attached directly to the skin 11 or intra-vascular cannulas may be inserted.

In addition, the glove 28 may be equipped with a finger opening 16 or multiple finger openings 16 (only one opening is illustrated) through which to access a pulse-oximetry point 35 or other instrumentation, as desired. The finger-tips of the gloves 28 may therefore be detachable, hinged or have any manner of opening 16, which either permanently or temporarily facilitates access. In fact the glove element 28 of the instant invention may also take the form of fingerless gloves, although this design is not preferred as it may result in unwanted heat-loss from the finger-tips.

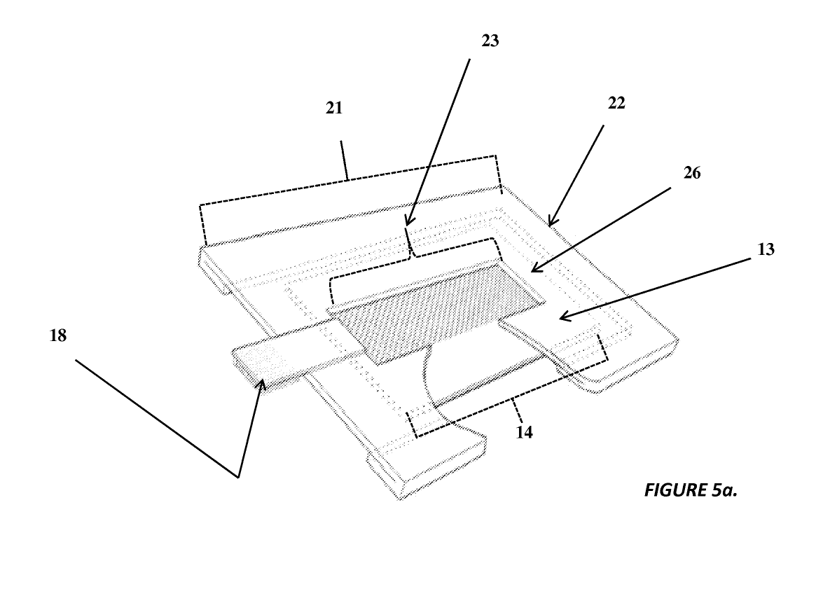

Turning now to the active heat element 14; in the instant device 10 the preferred method of providing a heat source to the extremities of the patient's limbs 19, 20, is via a self-contained, exothermic heat pack 14, illustrated in detail in FIG. 5. The heat pack 14 is preferentially of the type which is activated on contact with air, as, for example, that disclosed by Yim, U.S. Pat. No. 6,886,553, being a mixture of, “approximately 35-50% by weight of iron powder, 25-30% by weight of water, approximately 10-14% by weight of water retaining agent and approximately 4.5-6% by weight of salt.” Upon exposure to the air oxidation of the iron begins an exothermic reaction. Yim further provides guidelines for the range of heat radiating from the apparatus, defining it as between 26° C. and 30° C. and provides the rationale for this temperature regime as “in order to provide a level of heat suitable for therapeutic heating without danger of burn to human skin.” Yim further provides a temperature and time definition for the chemical heat emission of “between 26° C. and 30° C. for approximately 171 to 15 hours.”

Considering now the preferred method of deploying the heat element 14 as illustrated in FIG. 5; prior to activation the heat pack 14 is equipped with an air-tight seal (not illustrated) which is disclosed by folding back the protective cover 21. The integrity of the airtight seal is maintained in order to prevent premature and undesired activation of the heat element. 14. Once the air tight seal is breached, a vent 23 is opened, the exothermic reaction begins and the heat pack 14 generates heat. As the temperature generated by the heat pack 14 is derived from a chemical reaction which is proportional to the amount of air which is allowed to access the chemical mixture contained therein the heat pack 14 may, in an alternative configuration be equipped with a plurality of vents 23 (although only one is illustrated) with which to control the chemical reaction and thus the magnitude of heat output.

In an alternative arrangement, the entire garment pack is vacuum packed such that the heat elements are automatically activated when the pack 14 is exposed to air in the pre-surgical area. This configuration, which is within the scope of the instant invention, would clearly result in reduced garment complexity. However, although this arrangement is desirable due to the reduction in complexity of the garment structure 10, the inventors note that maintaining the integrity of a much larger sealed package through all aspects of shipping and distribution may prove problematic.

As illustrated in FIG. 5, the inventors believe it desirable that the heat element 14 is packaged, transported and deployed with an external cover 21. The cover 21 prevents both accidental transit damage to the heat pack 14 and also undesirable patient interference in the pre-operative environment. The heat pack cover 14 may beneficially be hinged 22 to the outer surface 13 of the layer of material 16. The hinge 22 orientation may also be varied, as desired.

In addition, the external cover, 21, may be insulated in order to retain heat, thereby improving the efficiency of the device. The material layer 21 which serves to protect the heat pack 14 in transit, is configured to encompass the outer surface of the heat pack 14 conferring insulative properties in order to preferentially direct the temperature towards the patient's skin 11, rather than allowing unwanted thermal egress into the operating room.

Although the inventors primarily intend the garments to be intervention-less, they note that deployment of heat packs 14 which are re-sealable such that a partial or total re-sealing of the heat element 14 results in a reduction or extinction of heat output increases garment versatility and is therefore a desirable feature. Therefore, if a lower temperature output is desired, the vent area 23 of the exothermic heat element 14, may be re-sealed, or partially re-sealed to reduce the thermal output. Conversely, the active vent area 23 of the exothermic heat element 14 may be re-opened in response to a reduction in patient core temperature. The instant device 10 therefore, is versatile in its responsiveness to variations in either environmental or patient core temperature resulting in greater flexibility in the operating room.

For patients undergoing shorter surgeries it may be preferable to allow some of the heat to dissipate to the environment, rather than confining it in proximity to the patient's skin 11. In this circumstance the cover 21 may be removed partially or in its entirety or be replaced by a cover 21 which has a smaller area than the heat pack 14 without departing from the spirit of the invention. However, releasing heat to the surgical environment is generally regarded as detrimental to the wellbeing of the surgical staff and this measure should be regarded as remedial.

As the preferred heating method is by exothermic chemical reaction, at least a portion of the cover 21 which substantially covers the heat element 14 is constructed to allow oxygen to enter the chemical heat pack 14. The cover 21 is therefore equipped with a breathable outer layer 26, which is dimensioned, to accommodate the specific oxygen requirement and thermal output of the heat pack 14 and may therefore cover a size substantially smaller, equal to or larger than the heat pack 14 as required. The breathable outer layer 26 may simultaneously display insulative properties which act to retain the heat between the cover 21 and the outer fabric surface 13 of the garment 10, thereby allowing the heat to be retained for the patient's benefit, within a greater surface area than is covered by the immediate heat element 14 itself.

Furthermore, the heat pack 14 may either be configured as an integral element of the garment 10 or may be configured to be positioned inside a pocket (not illustrated) on the garment 10; it may either be detachable, or be permanently attached to a surface of the garment 10 without departing from the spirit of the invention.

In the embodiment where the garment is equipped with a pocket, the heat pack 14 may be added to the garment 10 after the patient is clothed in the garment 10. The removable heat pack, pocket option garment, may be preferable during extended operations where surgery is likely to exceed the lifespan of the heat-packs. This allows the heat-packs to be replaced without interruption of the heat supply to the patient or without removal of the garment. Alternatively this option may also be considered preferable when the patient is of diminished responsibility.

The heat packs 14 may be deployed sequentially to augment the patient's core temperature in cases where lengthy surgical procedures are envisaged, or alternatively, it may be envisaged that additional heat sources 14 are bought on-line in response to observed reduction in the patient's core temperature.

Supplemental heat packs 14 may be also be advantageously deployed in the case where the patient's circulatory system has already been compromised, either prior to or during surgery, thus acting to increase the temperature of the extremities, and acting as a means of recovering compromised body core temperature.

The inventors conclude, however, that from a practical perspective, greater utility is provided where the heat-pack 14 is permanently attached to the palm 27 face of a glove, 10, and encapsulated by an insulated hinged 22 cover 21 and this represents a preferred embodiment of the invention.

For the further safety and protection of the patient, a visual means of providing the anesthesiologist with information about the patient's skin temperature may be provided. This fulfills two functions, serving to indicate to the surgical staff that heat is being generated effectively and also, provisionally, acting as a visual over-temperature indicator warning device. In keeping with the preferred disposable configuration of the device the inventors intend that the external surface of the garment be painted, printed or otherwise treated with thermal ink 24.

Thermal ink 24 is known as a means of monitoring temperature and a low cost thermal indicator is thereby integrated into the garment 10. In order for the ink treated area 24 to accurately reflect the actual output temperature as delivered to the patient's skin 11, the garment 10 is configured such that a thermal bridge (illustrated only as heat conductive means 25) is constructed between, on one end, the inner surface 12 of the garment 10 adjacent to the patient's skin, 11 and on the other end an area which is coated with the heat responsive ink 24. This mechanism reflects the temperature as it is delivered to the patient, rather than being indicative of the output temperature of the thermal source 14. Logically, the heat conductive filament 25 and ink treated area 24 may be extended circumferentially such that the heat sensitive ink treated portion 24 is visible at whatever the rotation or orientation of the patient's limb 19, 20. However, the ink treated portion or portions, 24 may be placed at any part of the garment 10, as desired, without departing from the spirit of the invention.

Thermal inks 24 are well known in many industries. Thermally reversible chromic inks such as those created by Colour Thermal, for example, may have a tolerance of 2% on temperature ranges between 30° C. and 80° C. This results in an effective temperature indicator scale of 1° C., which is appropriate for the purposes of establishing temperature measurements for the device 10. However, notwithstanding this preferred, simple method, any means may be used to indicate temperature without departing from the spirit of the invention.

As an alternative to thermal inks, LCD “temp-strips” may be used. These are commercially available, thermally reactive, self-adhesive, plastic strips which visually display the actual temperature by illuminating a numerical display. The accuracy of such devices is equivalent to the previously mentioned thermal inks at ±1° C.

Although the inventors have focused their attentions to the device as it pertains to the human anatomy, nothing in the invention precludes its use for animal husbandry uses and these are expressly claimed as being within the scope of the invention.

In summary, the inventors claim an intervention-less garment which is compatible with operating room practices and procedures, which acts to maintain patient euthermia, rather than responding to patient hypothermia. The goals of the device are to improve patient health and increase comfort levels, to reduce the duration of patient hospital stay and to improve the surgical environment through the removal of noisy, inefficient forced air warming devices.

Although the above descriptions illustrate certain preferred embodiments, they should be understood to be illustrative and inclusive of all alternatives, modifications and equivalents such as can be incorporated within the spirit and scope of the following claims.

### CLAUSES

### Clause 1:

The method of maintaining advantageously stabilized homeostatic parameters through normothermic maintenance in particular in the peri-surgical environment. In particular, the method of advantageously applying insulative garments over a small but disproportionately effective percentage of the BSA in order to counter environmentally and surgically induced hypothermia; The further method of maintaining normothermia using a disposable or re-usable, un-tethered, anatomically formed or substantially tube formed garment or plurality of garments preferentially configured to enclose the extremities and in particular the areas of the mammalian body which are the most effective loci of heat transfer.
The method, whereby the positive heating attributes of the system may be augmented through the use of additional insulative garments, which garments may be disposed about the patient's body to achieve greatest benefit to both patient and surgical field.
The method whereby the garment supports the placement of an intermittent compression device to any part of the upper or lower extremity for the purpose of enhancing compression of peripheral venous plexuses and optimizing return of distal blood to the core and acting as prophylaxis for deep venous prophylaxis. This method allows conforming garments to be applied without requiring removal for application of separate intermittent compression devices. The method of facilitating access for ancillary devices and interventions for the purpose of patient monitoring, intervention or diagnosis without requiring removal of the garment.

### Clause 2:

the system and apparatus of Clause 1 comprising a garment substantially designed to conform with the anatomy of the mammal being treated, being equipped with an internal and self-contained heat element, which apparatus preferentially consists of;
fabric gloves, mitts, slippers or socks designed to provide a passive thermal barrier against heat loss, which may be further improved through the use of cuff extensions, sleeves, greaves, or knee socks or stockings to further mitigate heat loss; an exothermic heat element incorporated into the structure of the garment so as to provide an integrated active and passive device for the support of euthermic temperature in medical patients.

### Clause 3:

the apparatus of Clause 2 where the garment fabric is constructed from substantially single-layered materials, which materials may be reinforced or supplemented by inserts or panels of fabrics whose properties are selected from a catalogue of desirable properties including, but not limited to, wicking, heat containment, heat dispersion, insulation, profiled fabrics and reinforcements;

### Clause 4:

the apparatus of Clause 2 whereby the garment is constructed from fabric sections which are attached by any appropriate means, including, but not limited to, gluing, stitching, laser-bonding or any other means as appropriate.

### Clause 5:

the apparatus of Clause 2 whereby the garment is constructed from a woven tube-like garment construction and where the garment fabric is substantially formed as a single, integrated structure.

### Clause 6:

the apparatus of Clause 2 where the heat element is one of a range selected from chemical exothermic reaction heaters, gel heat packs, or other heating means which are self-contained and requiring neither technician support, nor fixed electro-mechanical means, nor an external umbilical in order to function;

### Clause 7:

the apparatus of Clause 2 where heat distribution is enhanced through the use of heat conductive tracers, profiled fabric, or other means in order to optimize heat distribution from the heat element to the patient.

### Clause 8:

the apparatus of Clause 2 whereby the heat output of the device is monitored by visual temperature indicators, for example, by thermal-ink means.

### Clause 9:

the apparatus of Clause 2 whereby optional, pre-configured, openings in the garment support procedural, diagnostic or monitoring techniques, devices or instrumentation.

### Clause 10:

The apparatus of Clause 2 whereby the positive heating attributes of the system may be augmented through the use of additional insulative garments, which garments may be disposed about the patient's body to achieve greatest benefit to both patient and surgical field.

### Clause 11:

the apparatus of Clause 2 whereby the garment supports the placement of an intermittent compression device to any part of the upper or lower extremity for the purpose of enhancing compression of peripheral venous plexuses and optimizing return of distal blood to the core and acting as prophylaxis for deep venous prophylaxis. This allows conforming garments to be applied without requiring removal for application of separate intermittent compression devices.

### Clause 12:

The further method of Clause 1 whereby antibiotic prophylaxis is avoided.

1. A method of maintaining advantageously stabilized homeostatic parameters through normothermic maintenance, the method comprising applying insulative garments over a small but disproportionately effective percentage of the BSA in order to counter environmentally and surgically induced hypothermia.

2. The method as claimed in claim 1, wherein the method is implemented in a peri-surgical environment.

3. The method of maintaining normothermia, which method comprises use of a disposable or re-usable, un-tethered, anatomically formed or substantially tube formed garment or plurality of garments.

4. The method as claimed in claim 3, wherein the garments are configured to enclose the extremities.

5. The method as claimed in claim 4, wherein the extremities are areas of the mammalian body which are the most effective loci of heat transfer.

6. The method of augmenting the positive heating attributes of a patient system, which methods comprises the use of additional insulative garments, which garments may be disposed about the patient's body to achieve greatest benefit to both patient and surgical field.

7. The method according to claim 1, wherein the garment supports the placement of an intermittent compression device to any part of the upper or lower extremity for the purpose of enhancing compression of peripheral venous plexuses and optimizing return of distal blood to the core and acting as prophylaxis for deep venous prophylaxis.

8. The method according to claim 7, wherein the garments are conforming garments and may be applied without requiring removal for application of separate intermittent compression devices.

9. The method of facilitating access for ancillary devices and interventions for the purpose of patient monitoring, intervention or diagnosis without requiring removal of the garment.

10. The system and apparatus comprising a garment substantially designed to conform with the anatomy of the mammal being treated, being equipped with an internal and self-contained heat element.

11. The system and apparatus according to claim 10, which comprises fabric gloves, mitts, slippers or socks designed to provide a passive thermal barrier against heat loss.

12. The system and apparatus according to claim 11, which further comprise one or more of cuff extensions, sleeves, greaves, or knee socks or stockings to further mitigate heat loss.

13. The system and apparatus according to claim 10, further comprising an exothermic heat element incorporated into the structure of the garment so as to provide an integrated active and passive device for the support of euthermic temperature in medical patients.

14. The system and apparatus according to claim 10, wherein the garment fabric is constructed from substantially single-layered materials.

15. The system and apparatus according to claim 11, in which the materials may be reinforced or supplemented by inserts or panels of fabrics whose properties are selected from a catalogue of desirable properties including, one or more of wicking, heat containment, heat dispersion, insulation, profiled fabrics and reinforcements.

16. The system and apparatus according to claim 10, wherein the garment is constructed from fabric sections which are attached by any appropriate means, including, but not limited to, gluing, stitching, laser-bonding or any other means as appropriate.

17. The system and apparatus according to claim 10, wherein the garment is constructed from a woven tube-like garment construction.

18. The system and apparatus according to claim 10, wherein the garment fabric is substantially formed as a single, integrated structure.

19. The system and apparatus according to claim 10, wherein the source of heat is one of a range selected from chemical exothermic reaction heaters, gel heat packs, or other heating means.

20. The system and apparatus according to claim 19, in which the source of heat is self-contained and requiring neither technician support, nor fixed electro-mechanical means, nor an external umbilical in order to function.

21. The system and apparatus according to claim 10, wherein heat distribution is enhanced through the use of heat conductive tracers, profiled fabric, or other means in order to optimize heat distribution from the heat element to the patient.

22. The system and apparatus according to claim 10, wherein the heat output of the device is monitored by visual temperature indicators, for example, by thermal-ink means.

23. The system and apparatus according to claim 10, wherein pre-configured, openings are provided in the garment to support procedural, diagnostic or monitoring techniques, devices or instrumentation.

24. The system and apparatus according to claim 10, wherein the positive heating attributes of the system may be augmented through the use of additional insulative garments, which garments may be disposed about the patient's body to achieve greatest benefit to both patient and surgical field.

25. The system and apparatus according to claim 10, wherein the garment supports the placement of an intermittent compression device to any part of the upper or lower extremity for the purpose of enhancing compression of peripheral venous plexuses and optimizing return of distal blood to the core and acting as prophylaxis for deep venous prophylaxis.

26. The system and apparatus according to claim 10, wherein the conforming garments may be applied without requiring removal for application of separate intermittent compression devices.

27. The method according to claim 1, wherein the method is implemented without the use of antibiotic prophylaxis.
